(19) United States
(12) Patent Application Publication
(10) Pub. No.: US 2025/0020584 A1
(43) Pub. Date: Jan. 16, 2025

(54) SYSTEM AND METHOD FOR SPECTRAL-BASED DISEASE DETECTION

(71) Applicant: PATTERN COMPUTER, INC., Friday Harbor, WA (US)

(72) Inventors: Matthew Todd KEENER, Sammamish, WA (US); William Bradley HOLTZ, Bethesda, MD (US); Alejandro WOLF-YADLIN, Redmond, WA (US); Luca PION-TONACHINI, San Diego, CA (US)

(21) Appl. No.: 18/900,568

(22) Filed: Sep. 27, 2024

Related U.S. Application Data
(63) Continuation of application No. PCT/US2023/016386, filed on Mar. 27, 2023.
(60) Provisional application No. 63/325,076, filed on Mar. 29, 2022.

(51) Int. Cl. G01N 21/359; G16H 50/20

(52) U.S. Cl. CPC G01N 21/359; G16H 50/20; G01N 2201/08; G01N 2201/1296

(57) ABSTRACT

A system includes a light source that emits broadband light through a test cassette containing a fluid. A spectrometer disperses the broadband light, after passing through the test cassette, onto a plurality of pixel detectors of a detector array. A digitizer reads an array of raw spectral values from the detector array. A signal processing circuit deconvolves, based on a spectral response of each of the plurality of pixel detectors, the array of raw spectral values to generate an array of deconvolved spectral values; feeds the array of deconvolved spectral values into a trained machine-learning model to obtain an indication of the presence of one or more constituents in the fluid; and outputs the indication. The fluid may be human saliva or another biological fluid obtained from a patient. The indication may be used to diagnose the patient with a disease or condition.

### RELATED APPLICATIONS

This application is a Bypass continuation of International Application No. PCT/US2023/016386, filed on Mar. 27, 2023, which claims priority to U.S. Provisional Patent Application No. 63/325,076, filed on Mar. 29, 2022, the entirety of which is incorporated herein by reference.

### BRIEF DESCRIPTION OF THE FIGURES

FIG. 1 is a block diagram of a system for spectral-based disease detection, in an embodiment.

FIG. 2 is a block diagram of a system that is similar to the system of FIG. 1 except that it uses two spectrometers instead of one, in an embodiment.

FIG. 3 illustrates a method for processing a first and second arrays of raw spectral values, in an embodiment.

FIG. 4 illustrates a method for processing the first and second arrays of raw spectral values, in an embodiment.

FIG. 5 shows how the system of FIG. 2 can be implemented with fiber-optic components, in an embodiment.

FIG. 6 shows another way that the system of FIG. 2 can be implemented with fiber-optic components, in an embodiment.

FIG. 7 is a block diagram of a system that is similar to the system of FIG. 2 except that it uses two separate light beams instead of one, in an embodiment.

FIG. 8 illustrates how each pixel detector of FIG. 1, in the process of discretely sampling a continuous spectral density p(λ), effectively convolves the spectral density p(λ) with a sensitivity function ƒᵢ(λ), or kernel, that is unique to said each pixel detector.

FIG. 9 illustrates how light at one wavelength λⱼ contributes to several spectral values.

FIG. 10 is a functional diagram of a computational system that is one example of the signal processing circuit of FIG. 1, in embodiments.

### DETAILED DESCRIPTION

A biomarker is a measurable indicator of a disease or physical condition in an organism. The physical condition may be a normal biological process, a pathogenic process, or a response to a therapeutic intervention (e.g., a pharmacological response to a prescribed medication). For clinical purposes, biomarkers may be used to guide or narrow treatment options for a patient. More specifically, biomarkers may be used predictively (i.e., to predict clinical outcomes for the patient), diagnostically (i.e., to help diagnose the patient), or prognostically (i.e., to identify overall outcomes).

The spread of SARS-COV-2 (severe acute respiratory syndrome coronavirus-2, also referred to as a Covid-19 disease) has renewed interest to improve testing that can detect this disease and others. Currently, the most accurate diagnosis of SARS-Cov-2 virus uses polymerase chain reaction (PCR), such as quantitative reverse transcription PCR (RT-qPCR), which amplifies DNA and RNA sequences to make them easier to detect. Nasal swab tests using PCR-based detection are accurate, but have several limitations, including how the samples are handled (e.g., improper swabbing and storage), the requirement that sampling occurs during the acute phase, and a long testing time. For example, it can take 2 to 4 hours for PCR acquisition, and more than 12 hours for overall processing and handling. PCR machines are also large, expensive, and require technicians to operate properly.

Antigen tests are also now commonly used to detect SARS-Cov-2. Antigen tests identify the presence of a virus in nose and throat secretions by looking for proteins made by the virus (as opposed to the diagnostic test, which looks for genetic material directly). Advantageously, antigen tests take only 15 minutes, are inexpensive, and can be performed at-home without a medical professional or expensive equipment. However, antigen tests do not have the accuracy of PCR-based tests and are known for high rates of false negatives, especially for patients with a low viral load. Antigen tests may also give incorrect results due to improper handling (e.g., insufficient swabbing). They also require reagents, which can be difficult to produce and obtain in the middle of a pandemic.

More recently, light-based diagnosis techniques are being explored to combine the sensitivity and specificity of PCR-based testing with the low cost, high-speed, and scalability of antigen tests. Some of these light-based tests do not require reagents, thereby eliminating an important problem with PCR and antigen-based tests. These light-based tests perform spectroscopy (e.g., attenuated total reflection Fourier-transform infrared (ATR-FTIR) spectroscopy) on a sample obtained from a nasal swab or gargle to identify a spectral signature that is known to correlate with the presence of COVID-19.

The present disclosure comprises embodiments for spectroscopically detecting the presence of one or more constituents in a fluid sample. Specifically, the fluid sample is first measured to obtain an absorption spectrum. This absorption spectrum is then fed into a machine-learning model that is trained to identify one or more signatures in the absorption spectrum. Each signature is assumed to be unique to a corresponding constituent, and therefore the presence of a signature in the absorption spectrum indicates the presence of the corresponding constituent in the fluid sample. The machine-learning model outputs an indication (e.g., probability) of this presence.

Some of the present embodiments may be used with a biological fluid (e.g., blood, saliva, mucus, etc.) to rapidly diagnose the presence of a disease in the fluid. For example, when used with a small-volume sample of saliva, some of these embodiments can detect a SARS-COV-2 viral infection in under thirty seconds. Here, “small-volume” means that a typical human patient can non-invasively provide the sample with little effort (e.g., spitting into a cup) and minimal, if any, assistance. More generally, these embodiments can be adapted to detect any type of chemical or cellular biomarker obtained from any type of organism. Such biomarkers indicate a disease state or physiological state of the organism. Examples of such states include, but are not limited to, viral infections, bacterial infections, toxin-based diseases, cancers, heart conditions, or a lack thereof.

A medical worker (e.g., doctor, nurse, etc.) may use the outputted indication to diagnose the human patient with a disease or condition associated with the biomarker. However, the medical worker may also, based on the indication, diagnose the human patient as lacking the disease or condition. Thus, some of the present embodiments may be used to not only identify a patient as being ill, but also to determine if the patient is not ill. In either case, the medical worker may then provide an appropriate therapeutic intervention, if needed. For example, if the medical worker diagnoses the patient as having the disease, he or she may then prescribe, provide, or perform a therapeutic intervention for treating the disease. The medical worker may use additional information, and possible additional measured biomarkers, to decide on the therapeutic intervention (e.g., age, gender, blood pressure, temperature, etc.). Examples of therapeutic interventions include, but are not limited to, surgical procedures, non-surgical medical procedures, and prescriptions for one or more pharmaceutical drugs.

Some of the present embodiments operate with no chemical reagents, advantageously speeding up and simplifying sample preparation. Simplifying sample preparation improves accuracy by reducing errors that may arise during handling (e.g., gathering the sample and inserting it into the system). Eliminating chemical reagents also reduces the threat of supply chain disruption, as may arise during a pandemic response. Furthermore, by updating a machine-learning model, these embodiments can be quickly and easily adapted (e.g., via software updates) to detect a different disease or physiological state (e.g., a variant of a virus).

The combination of speed and lack of reagents makes at least some of the present embodiments ideal for use in real-time clearance applications. For example, some of the present embodiments may be used to screen airline passengers prior to boarding an airplane or entering an airport terminal. In a related application, some of the present embodiments may be used to test people who wish to access the interior of a building (e.g., a triage center, health-care facility, laboratory, arena, stadium, etc.). However, these embodiments may also be used to test other people in other situations, such as students who become sick during the day, patients who have already been admitted to a hospital or clinic, and individuals visiting an outpatient medical facility (e.g., a doctor's office).

Some of the present embodiments include, or use, one or more spectrometers, each of which disperses broadband light onto a plurality of pixel detectors forming a detector array. Associated with each pixel detector is a wavelength-dependent sensitivity function that is peaked at a center wavelength. Thus, while each pixel detector detects a small wavelength range of the broadband light (e.g., 0.1-1 nm), its electrical output is, for simplicity, associated with its center wavelength. Accordingly, associated with the detector array is a plurality of center wavelengths forming a one-to-one correspondence with the plurality of pixel detectors.

The machine-learning model accepts a plurality of spectral values as an input. Associated with each of these spectral values is a corresponding reference wavelength. However, the machine-learning model will likely have been trained with reference wavelengths that are different from the center wavelengths of a particular spectrometer. These differences arise from manufacturing tolerances during constructions of the spectrometers. Simply feeding the outputs of the pixel detectors directly into the machine-learning model can therefore degrade accuracy.

Some of the present embodiments solve this problem using a technique referred to herein as wavelength-shifting deconvolution. Like prior-art deconvolution techniques, wavelength-shifting deconvolution sharpens a measured spectrum by undoing the blurring effects of the sensitivity functions. However, unlike prior-art deconvolution techniques, wavelength-shifting deconvolution corrects the inputted spectral values for differences between the center wavelengths and reference wavelengths. Thus, wavelength-shifting deconvolution improves accuracy of the indications outputted by the machine-learning model by ensuring that the spectral values fed into the machine-learning model correctly indicate measurements at the reference wavelengths. Furthermore, wavelength-shifting deconvolution allows the same machine-learning model to be used across all units, regardless of variations in center wavelengths. Wavelength-shifting deconvolution can also be used to change the number of spectral values to match the number of inputs of the machine-learning model. Thus, with wavelength-shifting deconvolution, there is no need for the number of inputs to match the number of pixel detectors.

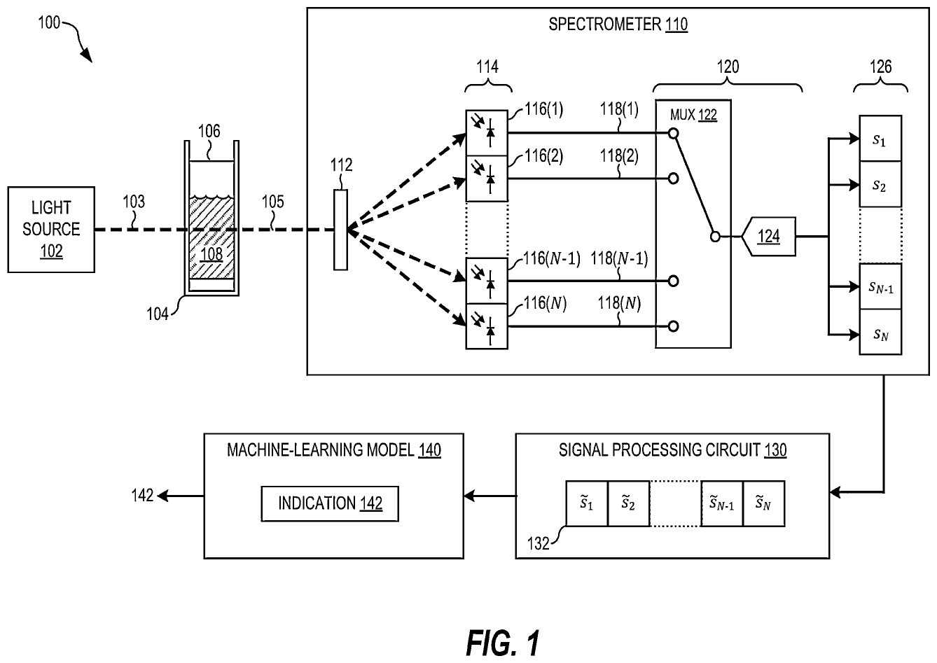

FIG. 1 is a block diagram of a system 100 for spectral-based disease detection. The system 100 includes a light source 102 that emits broadband light 103 through a fluid 108 confined within a test cassette 106. A portion of the broadband light 103 exiting the test cassette 106 is referred to as transmitted light 105. The system 100 also includes a cassette holder 104 that is shaped to receive the test cassette 106 and position the test cassette 106 such that the broadband light 103 enters the fluid 108 and the transmitted light 105 exits the fluid 108.

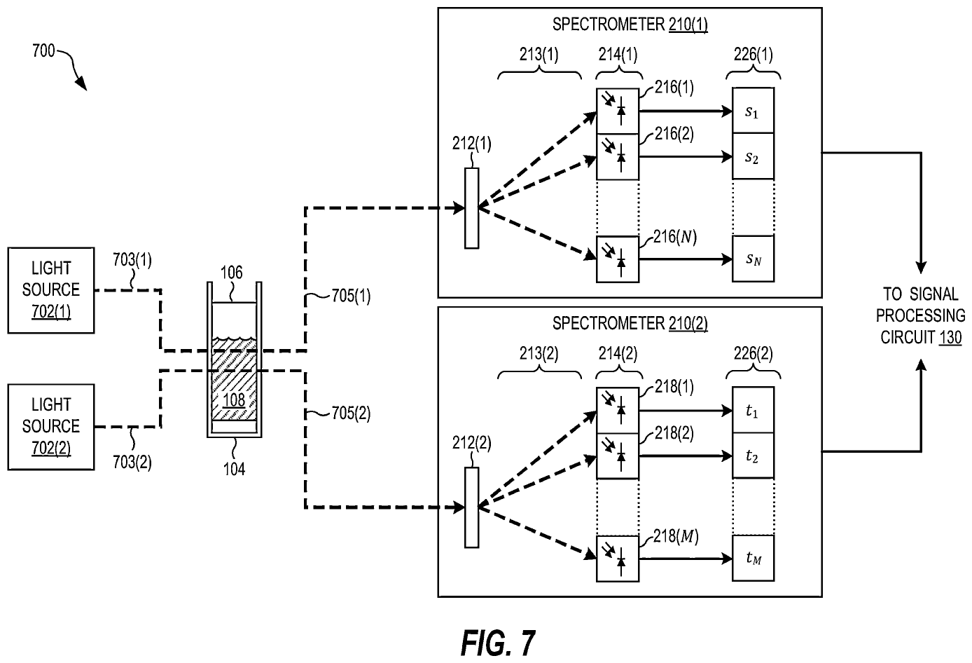

To enhance transmission of the broadband light 103 through the test cassette 106, the cassette holder 104 may include a pair of opposing optically transparent windows through which the broadband light 103 passes to enter and exit the test cassette 106. Alternatively, the cassette holder 104 may form apertures (e.g., holes) through which the broadband light 103 passes. Similarly, the test cassette 106 may be fabricated from a material that is at least partially transparent to the broadband light 103. For example, the test cassette 106 may be a glass cuvette, in which case the cassette holder 104 may be a cuvette holder. However, the test cassette 106 may be alternatively fabricated from another transparent material (e.g., plastic, crystal) without departing from the scope hereof. As discussed below with respect to FIG. 7, the test cassette 106 may be fabricated from multiple materials, as opposed to a single uniform material.

The system 100 also includes a spectrometer 110 that measures an absorption spectrum of the fluid 108. The fluid 108 includes one or more constituents, each having an absorption spectrum that is assumed to be unique and therefore can be used to identify the presence of the constituent in the fluid 108. The features of a constituent's absorption spectrum that make it unique are collectively referred to as a “signature”. Thus, some of the present embodiments identify the presence of a constituent by identifying its signature in the measured absorption spectrum. Features that contribute to a signature may include the presence of one or more absorption dips, the center wavelengths of these absorption dips, the widths of such absorption dips (e.g., a full-width at half maximum), and slopes of the spectrum within certain spectral bands (e.g., rising, falling, or flat).

Examples of constituents in the fluid 108 include microscopic organisms, such as archaea, bacteria, prokaryotes, and eukaryotes. These organisms need not be living when they are being measured by the system 100. Other constituents include viruses, prions, fragments of microorganisms (e.g., organelles), macromolecules (e.g., proteins, DNA, nucleic acids, carbohydrates, etc.), and organic compounds (e.g., small molecules and polymers). A constituent may have been produced by one or more types of microscopic organisms that are either present in the fluid 108 or were previously present (e.g., viral proteins). Alternatively, a constituent may have been generated by a host in response to the presence of a microscopic organism in the host (e.g., antibodies). The fluid 108 may alternatively or additionally contain other constituents without departing from the scope hereof.

The fluid 108 may also be created by dissolving a solid into a liquid solute, or by blending a solid with a base liquid. In some embodiments, the fluid 108 is replaced with a solid that is at least partially transparent to the broadband light 103 (e.g., a tissue sample). Thus, the present embodiments are not limited to fluids, i.e., some of the present embodiments may be used with gases instead.

The fluid 108 may be a sample obtained from a human patient. Examples of such samples include saliva, blood, mucus, and urine. Alternatively, the sample may be taken from a non-human animal, such as a household pet, farm animal, or zoo animal. However, the fluid 108 may be another kind of biological fluid without departing from the scope hereof. Furthermore, the fluid 108 need not be taken from a human being or animal. For example, the fluid 108 may be a sample taken from a body of water (e.g., lake, reservoir, ocean, etc.), water well, water processing facility, sewage processing facility, or sewer system.

To identify a signature of interest in the absorption spectrum, the broadband light 103 can cover a range of wavelengths that is wide enough to ensure that all features needed to determine the signature are measured. Similarly, the spectrometer 110 can be designed to operate over this wavelength range. In some embodiments, the wavelength range of the broadband light 103 spans at least a portion of the visible region of the electromagnetic spectrum (e.g., approximately 350-750 nm in air or vacuum). In other embodiments, the wavelength range spans at least a portion of the infrared region of the electromagnetic spectrum. For example, the wavelength range may span the near-infrared region of 750-1400 nm. Alternatively, the wavelength range may span 900-1700 nm. In some embodiments, the wavelength spans a portion of both the visible and infrared regions (e.g., 600-1000 nm). The wavelength range may at least partially span one or more other regions of the electromagnetic spectrum (e.g., ultraviolet, mid-infrared, far-infrared, etc.) without departing from the scope hereof. For technical reasons that are described in more detail below, a wavelength range spanning more than a factor of two may be best detected with two separate spectrometers operating in parallel. However, there is no such limitation on the wavelength range of the broadband light 103. Thus, in some embodiments, the wavelength range spans more than a factor of two (e.g., 350-1700 nm).

The light source 102 uses one or more light-generating devices to create the broadband light 103 as a single optical beam. The broadband light 103 may be incoherent, in which case the light source 102 may include a lamp, discharge tube, or LED source. Incoherent broadband light 103 may be white light (or similarly colored light) that contains all wavelengths in the wavelength range. Alternatively, the broadband light 103 may be coherent, in which case the light source 102 may include a laser. Coherent broadband light 103 may be quasi-white light (or similarly quasi-colored light) having discrete frequency components that are equally spaced in frequency. Such quasi-white light may be generated, for example, by an ultrafast pulsed laser (e.g., a modelocked femtosecond titanium-sapphire laser or fiber laser). The wavelength range of this quasi-white light may be further extended using nonlinear optical techniques, such as four-wave mixing in a nonlinear crystal or optical medium (e.g., a photonic crystal fiber). As described in more detail below, the light source 102 may combine the outputs of two or more light-generating devices. These two or more light-generating devices may be incoherent light sources, coherent light sources, or a combination of coherent and incoherent light sources.

The broadband light 103 exits the fluid 108 and test cassette 106 as transmitted light 105 that is subsequently processed by the spectrometer 110. The spectrometer 110 includes a dispersive element 112 that disperses the transmitted light 105 onto a plurality of N pixel detectors 116(1) . . . 116(N) arranged as a detector array 114, where N is an integer greater than 1. Each pixel detector 116(i) of the detector array 114 outputs a corresponding photocurrent 118(i) that is approximately linearly proportional to the irradiance of the portion of the transmitted light 105 that is incident on the pixel detector 116(i). Since each pixel detector 116(i) measures light power, the spectrometer 110 may also be referred to as a spectroradiometer. While the dispersive element 112 is shown in FIG. 1 as a transmissive diffraction grating, the dispersive element 112 may alternatively be a reflective diffraction grating, a prism, or another type of optical component that spatially disperses polychromatic light. The spectrometer 110 may include one or more other components that are not shown in FIG. 1, such as a slit, lenses, mirrors (e.g., for beam steering), filters, and so on. In other embodiments, the spectrometer 110 includes a scanning monochromator. In these embodiments, only one photodetector is needed.

The detector array 114 may be a one-dimensional array, as shown in FIG. 1. Alternatively, the detector array 114 may be a two-dimensional array (e.g., a CMOS or CCD image sensor). In this case, the outputs of the pixels of each row (or column) may be integrated to obtain a single value. Each pixel detector 116(i) detects a spectral subrange of the transmitted light 105 according to a spectral sensitivity function ƒᵢ(λ). The sensitivity function ƒᵢ(λ) is also referred to as a kernel function, blur function, or bandwidth function. Typically, each sensitivity function ƒᵢ(λ) is peaked at a center wavelength λᵢ^{(c)}, which is the wavelength at which the photocurrent 118(i) outputted by the pixel detector 116(i) is greatest. Thus, the pixel detectors 116(1) . . . 116(N) form a one-to-one correspondence with a plurality of center wavelengths λ₁^{(c)} . . . λ_{N}^{(c)}. Additional details about the spectral sensitivity function ƒᵢ(λ) are presented below.

Each pixel detector 116(i) is shown in FIG. 1 as a photodiode. Each photodiode may be fabricated from materials such that the photodiode has a high responsivity at the spectral subrange it detects. Examples of such materials include silicon (Si), gallium arsenide (GaAs), indium gallium arsenide (InGaAs), and germanium (Ge). When detecting low light levels, each photodiode may be an avalanche photodiode. In this case, the detector array 114 may be a multi-pixel photon counter or silicon multiplier. Each pixel detector 116(i) may be another type of light detector, photodetector, or photosensor without departing from the scope hereof.

The spectrometer 110 also includes a digitizer 120 that reads an array 126 of raw spectral values s₁ . . . s_{N} from the detector array 114. Specifically, the digitizer 120 samples each photocurrent 118(i) and outputs a corresponding raw spectral value sᵢ that is a digital representation of the sample. In the example of FIG. 1, the digitizer 120 includes a multiplexer (MUX) 122 that connects one of the photocurrents 118 to the input of an analog-to-digital converter (ADC) 124. In this case, the digitizer 120 digitizes the photocurrents 118 sequentially (i.e., one at a time). Alternatively, the digitizer 120 may contain N ADCs, in which case each photocurrent 118(i) is digitized by its own dedicated ADC. While dedicated ADCs allow all of the photocurrents 118(1) . . . 118(N) to be processed simultaneously, there may be thousands of pixel detectors 116 (i.e., N>1000), or more, and having so many dedicated ADCs may be prohibitively expensive and complex to build. As an alternative, the digitizer 120 may include a plurality of ADCs (e.g., four ADCs integrated into a single quad ADC chip) and a plurality of multiplexers 122 that are wired such that each of the pixel detectors 116 is sampled by one of the plurality of ADCs. The digitizer 120 may be alternatively configured without departing from the scope hereof. While FIG. 1 shows the digitizer 120 as part of the spectrometer 110, the digitizer 120 may alternatively be separate from the spectrometer 110.

In FIG. 1, the order of the raw spectral values s₁ . . . s_{N} in the array 126 matches that of the pixel detectors 116(1) . . . 116(N) and therefore that of the center wavelengths λ₁^{(c)} . . . λ_{N}(c). Maintaining this order may simplify subsequent signal processing. However, there is no requirement for the order of the raw spectral values s₁ . . . s_{N} provided that the order is known so that the subsequent signal processing can be implemented properly.

The spectrometer 110 may also be operated as a spectrophotometer. Specifically, the system 100 may be operated twice: once with the fluid 108 in the test cassette 106 and once without. The measurement obtained without the fluid 108 serves as a baseline that can be subtracted from the measurement obtained with the fluid 108 to cancel out systematic effects that do not arise from the fluid 108 itself. Such systematic effects include the non-uniform absorption spectrum of the test cassette 106, the cassette holder 104, and other optics between the light source 102 and spectrometer 110. Another systematic effect is the non-uniformity of the broadband light 103 (i.e., the broadband light 103 may not be perfectly white).

The system 100 also includes a signal processing circuit 130 that deconvolves, based on the spectral sensitivity function ƒᵢ(λ) of each of the pixel detectors 116, the array 126 of raw spectral values s₁ . . . s_{N} to generate an array 132 of deconvolved spectral values \tilde{s}₁ . . . \tilde{s}_{N}. As described in more detail below, the spectral sensitivity functions ƒ₁(λ) . . . ƒ_{N}(λ) may be combined into a kernel matrix that is multiplied with the array 126 to obtain the array 132.

The signal processing circuit 130 also feeds the array 132 of deconvolved spectral values \tilde{s}₁ . . . \tilde{s}_{N} into a machine-learning model 140 that transforms the array 132 into an indication 142 of the presence of one or more constituents in the fluid 108. The indication 142 may include, for example, a probability of the presence (or absence) of a particular constituent in the fluid 108. This probability may be determined by the machine-learning model 140 by comparing the array 132 to a known signature of the particular constituent. Thus, the probability quantifies the likelihood of the signature appearing in the array 132. In some embodiments, the indication 142 includes a plurality of probabilities, each indicating the presence (or absence) of a corresponding one of a plurality of constituents in the fluid 108.

The machine-learning model 140 may be any type of machine-learning model known in the art. Examples include, but are not limited to, an artificial neural network, a support vector machine, a decision tree, a random forest, a regression model, and combinations thereof. The machine-learning model 140 is trained to recognize one or more constituents using supervisory data in which the known signature of one or more constituents is labeled. In this sense, the signature is “known” to the machine-learning model 140.

In some embodiments, the machine-learning model 140 is stored in, and executed by, the signal processing circuit 130 or another circuit of the system 100. In other embodiments, the machine-learning model 140 is provided by a third party and is therefore not part of the system 100. For example, the machine-learning model 140 may be implemented on a remote computer server that is operated by the third party (e.g., Platform-as-a-Service). In these embodiments, the signal processing circuit 130 (or another component of the system 100) transmits the array 132 to the remote computer server for processing with the machine-learning model 140 (e.g., via the Internet). The system 100 then receives the indication 142 from the remote computer server. Use of the remote server may be advantageous when the computational resources needed to execute the machine-learning model 140 exceed what the signal processing circuit 130 can provide.

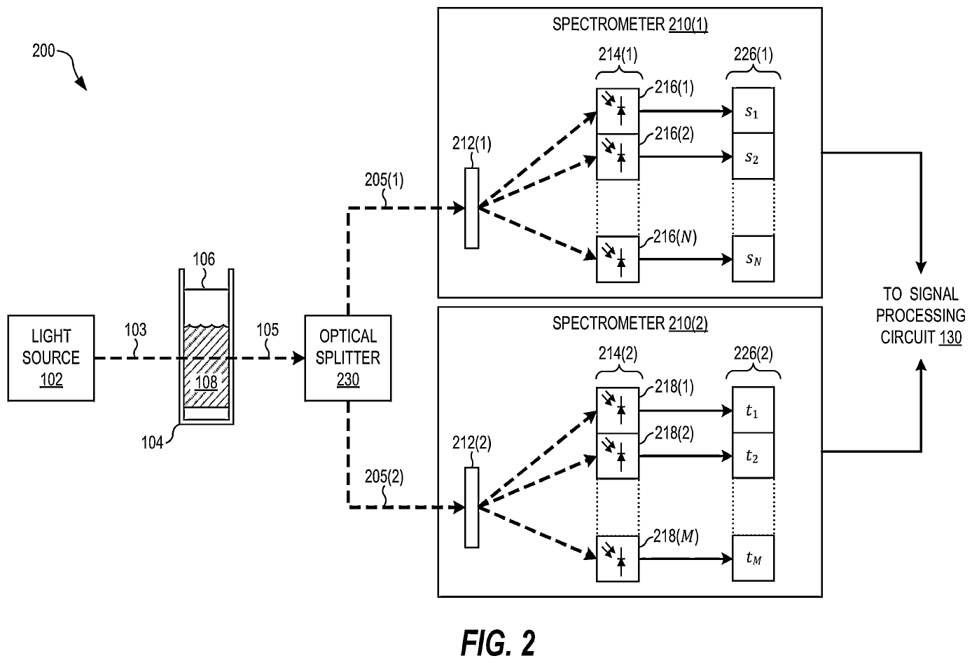

FIG. 2 is a block diagram of a system 200 that is similar to the system 100 of FIG. 1 except that it uses two spectrometers instead of one. Specifically, the system 200 includes an optical splitter 230 that splits the transmitted light 105 into a first portion 205(1) and a second portion 205(2). The system 200 includes a first spectrometer 210(1) that processes the first portion 205(1) and a second spectrometer 210(2) that processes the second portion 205(2). Each of the spectrometers 210(1) and 210(2) operates similarly to the spectrometer 110 of FIG. 1 and therefore may be configured in any manner described above for the spectrometer 110. The spectrometers 210(1) and 210(2) may process the respective portions 205(1) and 205(2) simultaneously.

The first spectrometer 210(1) includes a first dispersive element 212(1) that disperses the first portion 205(1) onto a plurality of N pixel detectors 216(1) . . . 216(N) arranged as a first detector array 214(1). Similarly, the second spectrometer 210(2) includes a second dispersive element 212(2) that disperses the second portion 205(2) onto a plurality of M pixel detectors 218(1) . . . 218(M) arranged as a second detector array 214(2), where M is an integer greater than 1. Furthermore, M may be equal to, less than, or greater than N. The dispersive elements 212(1) and 212(2) are similar to the dispersive element 112 of FIG. 1, the detector arrays 214(1) and 214(2) are similar to the detector array 114 of FIG. 1, and the pixel detectors 216 and 218 are similar to the pixel detectors 116 of FIG. 1.

The analog outputs of the first detector array 214(1) are digitized to create a first array 226(1) of raw spectral values s₁ . . . s_{N}. Similarly, the analog outputs of the second detector array 214(2) are digitized to create a second array 226(2) of raw spectral values t₁ . . . t_{M}. For clarity in FIG. 2, digitizers are not shown. The arrays 226(1) and 226(2) are then fed to the signal processing circuit 130 of FIG. 1 for merging, deconvolving, and subsequent processing by the machine-learning model 140 of FIG. 1. Thus, the system 200 generates the indication 142 based on both of the arrays 226(1) and 226(2), as opposed to just the one array 126 of FIG. 1.

As compared to the system 100 of FIG. 1, the system 200 advantageously improves accuracy when the wavelength range of the broadband light 103 is particularly large. For example, it is known in the art that consecutive diffraction orders of a grating overlap when the wavelength range of the incident light is large. In this case, different wavelengths will be incident on the same pixel detector 116. The system 200 solves this problem by allocating different wavelength ranges to the spectrometers 210(1) and 210(2). Specifically, the first spectrometer 210(1) operates over a first spectrometer wavelength range and the second spectrometer 210(2) operates over a second spectrometer wavelength range that is different from the first spectrometer wavelength range. For example, the first spectrometer 210(1) may operate with infrared light while the second spectrometer 210(2) operates with visible light. The wavelength range of the broadband light 103 may span both of the first and second spectrometer wavelength ranges. The first and second spectrometer wavelength ranges may at least partially overlap or not overlap at all.

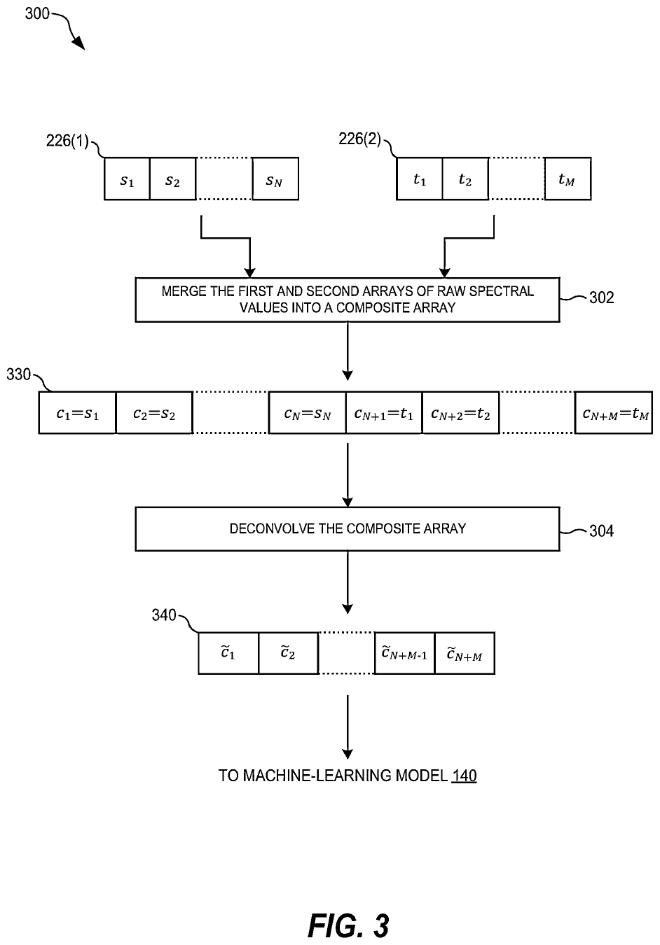

FIG. 3 illustrates a method 300 for processing the first array 226(1) of raw spectral values s₁ . . . s_{N} and the second array 226(2) of raw spectral values t₁ . . . t_{M}. The method 300 generates a deconvolved array 340 that is fed to the machine-learning model 140 of FIG. 1, which transforms the deconvolved array 340 into the indication 142 as described above. The method 300 may be performed by the signal processing circuit 130 of FIG. 1.

In the block 302 of the method 300, the arrays 226(1) and 226(2) are merged into a composite array 330 which has N+M raw spectral values denoted c₁ . . . c_{N+M}. As shown in FIG. 3, the composite array 330 may be generated by appending the second array 226(2) to the first array 226(1). Alternatively, the first array 226(1) may be appended to the second array 226(2). However, there is no requirement that the order of the raw spectral values s₁ . . . s_{N} or the order of the raw spectral values t₁ . . . t_{M} be maintained in the composite array 330. Thus, the composite array 330 may be generated by interleaving the arrays 226(1) and 226(2) or another merging technique. The composite array 330 may be sorted by the center wavelengths λ₁^{(c)} . . . λ_{N}^{(c)} of the pixel detectors 216 and the center wavelengths λ₁^{(c)} . . . λ_{M}^{(c)} of the pixel detectors 218. However, the composite array 330 may by sorted in another manner, or unsorted, without departing from the scope hereof.

In the block 304, the composite array 330 is deconvolved to generate the deconvolved array 340 of deconvolved spectral values \tilde{c}₁ . . . c_{N+M}. Any deconvolution method known in the art may be used, such as frequency domain deconvolution (also known as Fourier deconvolution or Fourier self-deconvolution) and spatial domain deconvolution. Examples of the latter include, but are not limited to, differential operator methods, higher-order statistical methods, maximum entropy deconvolution, and Bayesian deconvolution.

Deconvolution is frequently posed as an inversion problem. Since such inversions may be unstable, smoothing techniques may be used to improve stability. Examples of such smoothing techniques include, but are not limited to, interpolations, moving averages, and exponential smoothing. Another class technique used to improve stability is regularization. Examples of regularization include, but are not limited to, Tikhonov regularization, Huber Markov regularization, discrete cosine transform regularization, and wavelet transform regularization. More details about regularization are presented below. If the inversion is sufficiently stable, then a pseudo-inverse matrix can be found. This pseudo-inverse matrix is then simply multiplied by the composite array 330 to obtain the deconvolved array 340.

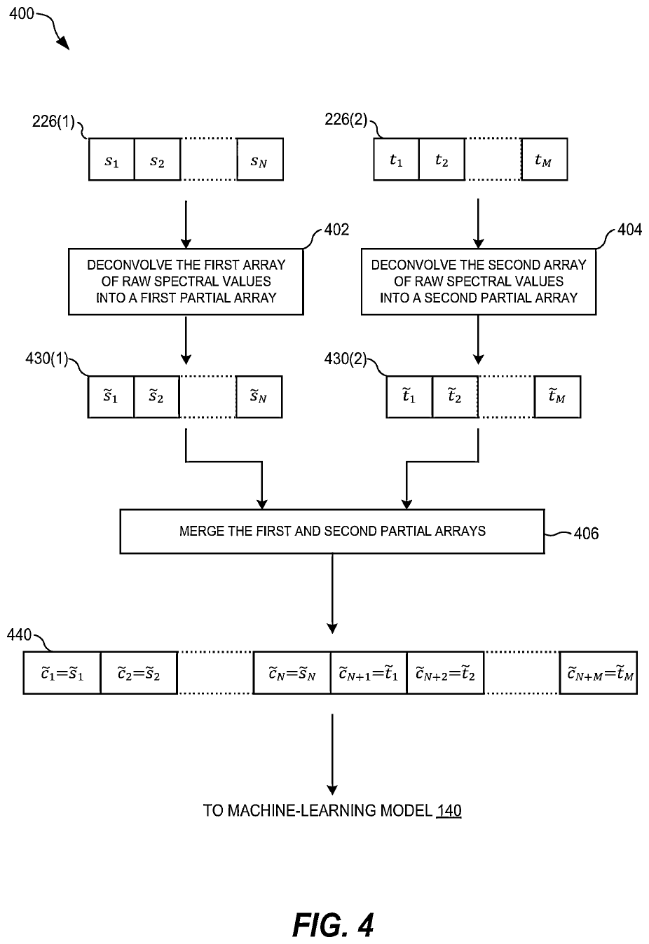

FIG. 4 illustrates a method 400 for processing the first array 226(1) of raw spectral values s₁ . . . s_{N} and the second array 226(2) of raw spectral values t₁ . . . t_{M}. The method 400 is similar to the method 300 of FIG. 3 in that it generates a deconvolved array 440 that is fed to the machine-learning model 140 of FIG. 1. The method 400 may also be performed by the signal processing circuit 130 of FIG. 1.

In the block 402, the first array 226(1) is deconvolved to generate a first partial array 430(1) of deconvolved spectral values \tilde{s}₁ . . . \tilde{s}_{N}. In the block 406, the second array 226(2) is deconvolved into a second partial array 430(2) of deconvolved spectral values \tilde{t}₁ . . . \tilde{t}_{M}. Each of the blocks 402 and 404 is similar to the block 304 of the method 300. In the block 406, the partial arrays 430(1) and 430(2) are merged to generate the deconvolved array 440. The block 406 is similar to the block 302 of FIG. 3.

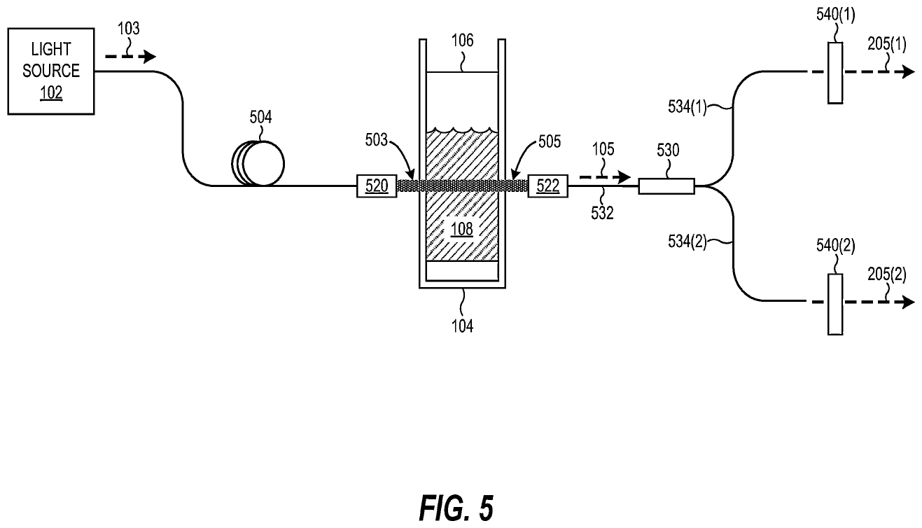

FIG. 5 shows how the system 200 of FIG. 2 can be implemented with fiber-optic components. In FIG. 5, broadband light 103 emitted by the light source 102 propagates along an optical fiber 504. A first free-space coupler 520 couples the broadband light 103 into a broadband optical beam 503 that propagates through the fluid 108 and exits the fluid 108 as a transmitted optical beam 505. A second free-space coupler 522 couples the transmitted optical beam 505 into an input optical fiber 532. The portion of the transmitted optical beam 505 that is coupled into the optical fiber 532 is an example of the transmitted light 105 shown in FIGS. 1 and 2. A fiber-optic splitter 530 splits this transmitted light 105 into the first portion 205(1) and the second portion 205(2). The fiber-optic splitter 530 is one example of the optical splitter 230 shown in FIG. 2. The first portion 205(1) propagates along a first output optical fiber 534(1) and the second portion 205(2) propagates along a second output optical fiber 534(2). The first output optical fiber 534(1) may end at the first spectrometer 210(1) to deliver the first portion 205(1) of the transmitted light 105 to the first spectrometer 210(1). Similarly, the second output optical fiber 534(2) may end at the second spectrometer 210(2) to deliver the second portion 205(2) of the transmitted light 105 to the second spectrometer 210(2).

The fiber-optic splitter 530 may be a fused biconical taper splitter, a planar lightwave circuit splitter, or another type of fiber-optic splitter known in the art. Accordingly, the fiber-optic splitter 530 may be an inline device in which the optical fibers 532, 534(1), and 534(2) are connectorized pigtails. Such an inline device with pigtails is also known as a fiber-optic Y cable. In other embodiments, the fiber-optic splitter 530 is a free-space optical splitter that uses free-space-to-fiber-optic couplers (e.g., the couplers 520 and 522 in FIG. 5) to couple light from the optical fiber 532 and to the optical fibers 534(1) and 534(2).

In some embodiments, the optical splitter 230 of FIG. 2 (e.g., the fiber-optic splitter 530 of FIG. 5) splits the transmitted light 105 based on wavelength such that the portions 205(1) and 205(2) have different wavelength ranges. For example, the optical splitter 230 may be a wavelength division multiplexer. As another example, the optical splitter 230 may use a hot mirror to reflect infrared light and transmit visible light (or a cold mirror that transmits infrared light and reflects visible light). The reflected infrared light serves as one of the portions 205(1) and 205(2) while the transmitted visible light serves as the other of the portions 205(2) and 205(2).

In some embodiments, the optical splitter 230 of FIG. 2 does not split the transmitted light 105 based on wavelength. For example, the optical splitter 230 may be a broadband beamsplitter (e.g., plate, cube, or fiber-optic). In this case, the portions 205(1) and 205(2) have the same, or similar, wavelength ranges. To prevent overlap of consecutive diffraction orders in the first spectrometer 210(1), a first filter 540(1) may be used to filter the first portion 502(1) to block wavelengths that lie outside of the first spectrometer wavelength range. Similarly, a second filter 540(2) may be used to filter the second portion 502(2) to block wavelengths that lie outside of the second spectrometer wavelength range. Each of the filters 540(1) and 540(2) may be an absorptive filter (e.g., a piece of colored glass) or an interference filter (e.g., a hot or cold mirror). Each of the filters 540(1) and 540(2) may be a longpass filter, a bandpass filter, or a shortpass filter. Each of the filters 540(1) and 540(2) may be another type of filter without departing from the scope hereof.

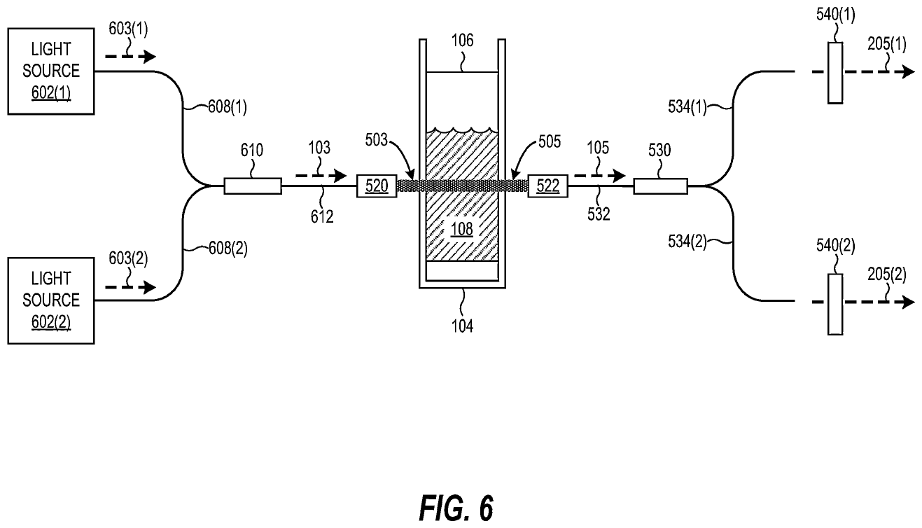

FIG. 6 shows another way that the system 200 of FIG. 2 can be implemented with fiber-optic components. In FIG. 6, a first light source 602(1) emits first broadband light 603(1) into a first optical fiber 608(1) and a second light source 602(2) emits second broadband light 603(2) into a second optical fiber 608(2). A fiber-optic combiner 610 combines the broadband lights 603(1) and 603(2) into the broadband light 103, which propagates along an optical fiber 612 to the free-space coupler 520. The light sources 602(1) and 602(2), optical fibers 608(1) and 608(2), and fiber-optic combiner 610 are collectively one example of the light source 102 shown in FIGS. 1 and 2. Since an optical combiner may be formed from an optical splitter operating in reverse, the fiber-optic combiner 610 may be similar to the fiber-optic splitter 530.

The implementation shown in FIG. 6 may be used when the wavelength range of the broadband light 103 needs to be wider than what any single light source can generate. For example, the first light source 602(1) may be a broadband infrared source while the second light source 602(2) may be a broadband visible light source. In this case, the broadband light 103 has a wavelength range that spans the visible and infrared. The broadband lights 603(1) and 603(2) may have wavelength ranges that partially overlap. Alternatively, the broadband lights 603(1) and 603(2) may have wavelength ranges that do not overlap. In one embodiment, the broadband lights 603(1) and 603(2) have respective first and second wavelength ranges that are similar to the first and second spectrometer wavelength ranges.

In other embodiments, the light sources 602(1) and 602(2) emit the broadband lights 603(1) and 603(2) as free-space optical beams. In these embodiments, the free-space coupler 520 and optical fibers 608(1), 608(2), and 612 are not needed and the fiber-optic combiner 610 may be replaced with a free-space optical beam combiner. Similarly, when the fiber-optic splitter 530 is replaced with a free-space splitter, the free-space coupler 522 and optical fibers 532, 534(1), and 534(2) are not needed.

FIG. 7 is a block diagram of a system 700 that is similar to the system 200 of FIG. 2 except that it uses two separate light beams instead of one. Specifically, the system 700 includes a first light source 702(1) that emits a first broadband optical beam 703(1) that propagates through the fluid 108. The system 700 also includes a second light source 702(2) that emits a second broadband optical beam 703(2) that propagates through the fluid 108. A portion of the first broadband optical beam 703(1) exiting the test cassette 106 is referred to as a first transmitted optical beam 705(1). Similarly, a portion of the second broadband optical beam 703(2) exiting the test cassette 106 is referred to as a second transmitted optical beam 705(2). As indicated in FIG. 7, the optical beams 703(1) and 703(2) pass through the fluid 108 as two spatially separate and distinct beams of light. Thus, the optical beams 703(1) and 703(2) do not spatially overlap anywhere within the volume of space occupied by the fluid 108.

Similar to FIG. 6, the system 700 uses two light sources to measure the absorption spectrum of the fluid 108 over a wavelength range that is wider than what any single light source can generate. However, the system 700 is simpler than what is shown in FIG. 6 since no optical combiner (e.g., the fiber-optic combiner 610 of FIG. 6) is needed. The system 700 is also simpler than the system 200 of FIG. 2 since no optical splitter (e.g., the optical splitter 230 of FIG. 2 or the fiber-optic splitter 530 of FIG. 5) is needed.

Another advantage of the system 700 is that keeping the optical beams 703(1) and 703(2) spatially separate prevents light contamination therebetween. For example, in FIG. 6 consider when the first broadband light 603(1) has a wavelength range similar to that of the first spectrometer 210(1) and the second broadband light 603(2) has a wavelength range similar to that of the second spectrometer 210(2). In this case, the fiber-optic splitter 530 would ideally split the transmitted light 105 such that (i) the first portion 205(1) only contains light that originated from the first broadband light 603(1) and (ii) the second portion 205(2) only contains light that originated from the second broadband light 603(2). However, due to imperfect performance of the fiber-optic splitter 530, some cross-contamination light originating from the second broadband light 603(2) will be present in the first portion 205(1). This cross-contamination light may degrade the sensitivity or accuracy of the optical spectrometer 210(1) due to overlap of consecutive orders, as described above. Similar arguments apply for cross-contamination light present in the second portion 205(2). Reducing this cross-contamination therefore light not only improves the performance of the spectrometers 210(1) and 210(2) but it also reduces the filtering requirements of the filters 540(1) and 540(2).

When used with the system 700, the test cassette 106 may be fabricated from two or more different materials having different transmission properties. For example, the test cassette 106 may include a first material through which the first broadband optical beam 703(1) (and not the second broadband optical beam 703(2)) passes and a second material through which the second broadband optical beam 703(2) (and not the first broadband optical beam 703(1)) passes. The first material may be selected for high transmission at the wavelengths of the first broadband optical beam 703(1). Similarly, the second material may be selected for high transmission at the wavelengths of the second transmitted optical beam 703(1). Thus, the use of multiple different materials can help increase transmission at all of the wavelengths detected by the spectrometers 210(1) and 210(2), as compared to using a single material that may have high transmission at some, but not all, of these wavelengths.

While the spectrometers 210(1) and 210(2) may operate simultaneously, they may alternatively be operated sequentially. For example, in FIG. 7 the first light source 702(1) and first spectrometer 210(1) may be operated while the second light source 702(2) does not emit the second broadband optical beam 703(2) (e.g., the second light source 702(2) may be turned off or shuttered). The first spectrometer 210(1) then measures the first transmitted optical beam 705(1) without cross-contamination light from the second broadband optical beam 703(2). After the first array 226(1) is created, the first light source 702(1) is turned off while the second light source 702(2) is turned on. The second spectrometer 210(2) then measures the second transmitted optical beam 705(2) without cross-contamination light from the first broadband optical beam 703(1). This sequential operation may also be implemented with the system 200 by sequentially operating the light sources 602(1) and 602(2) of FIG. 6.

Any of the present embodiments that work with two spectrometers (e.g., the system 200 of FIG. 2, the system 700 of FIG. 7, etc.) may be expanded to work with three or more spectrometers. Increasing the number of spectrometers may be useful when the wavelength range extends beyond what two spectrometers can cover. For example, in one embodiment the system 200 of FIG. 2 further includes a third spectrometer that operates in parallel to the first spectrometer 220(1) and the second spectrometer 220(2). The third spectrometer outputs a third array of raw spectral values similar to the first array 226(1) and the second array 226(2). The third spectrometer may operate over a third spectrometer wavelength range that at least partially covers one or both of the first spectrometer wavelength range and the second spectrometer wavelength range. Alternatively, the third spectrometer wavelength range may not cover any of the first spectrometer wavelength range and the second spectrometer wavelength range.

The signal processing circuit 130 may be adapted to process three or more arrays of raw spectral values. Similarly, the methods 300 and 400 may be similarly to operate with three or more arrays of raw spectral values. For example, in FIG. 3 the block 302 may be expanded to merge the third array with the arrays 226(1) and 226(2). In FIG. 4, the third array of raw spectral values may be deconvolved into a third partial array. The block 406 may then be expanded to merge this third partial array with the first and second partial arrays.

### Wavelength-Shifting Deconvolution

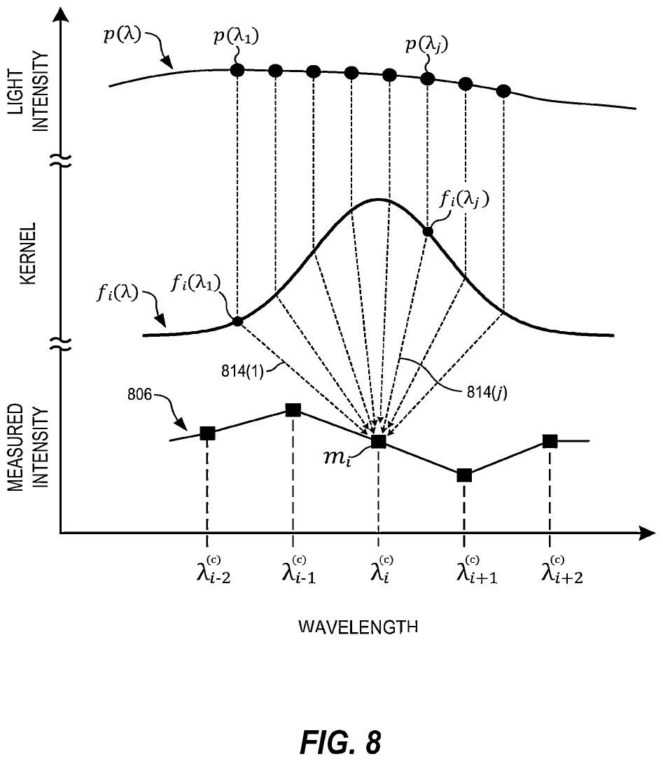

FIG. 8 illustrates how each pixel detector 116(i), in the process of discretely sampling a continuous spectral density p(λ), effectively convolves the spectral density p(λ) with the sensitivity function ƒᵢ(λ), or kernel, that is unique to said pixel detector 116(i). The spectral density p(λ) is indicated in FIG. 8 by a solid line while values of the spectral density p(λ) at several discrete wavelengths are indicated by circles. For clarity, only two of these values are labeled. Specifically, the value of the spectral density p(λ) at a first wavelength λ₁ is indicated by p(λ₁) and the value of the spectral density p(λ) at a j^{th} wavelength λⱼ is indicated by p(λⱼ).

The kernel ƒᵢ(λ) is shown in FIG. 8 having a Gaussian profile that is centered at the center wavelength λᵢ^{(c)} associated with the pixel detector 116(i). The Gaussian profile has a standard deviation denoted σ, and therefore a half width at half maximum (HWHM) approximately equal to 1.20. The HWHM is also known as the resolving resolution. In embodiments, the resolving resolution of each pixel detector 116(i) is between 0.2 and 3 nm. However, the resolving resolution of each pixel detector 116(i) may be greater than 0.2 nm or less than 3 nm without departing from the scope hereof. The resolving resolutions of the pixel detectors 116(1) . . . 116(N) typically vary with wavelength λ, and therefore are not necessarily identical. The kernel ƒᵢ(λ) may alternatively have a different “peaked” profile, such as a Lorentzian profile, the middle peak of a sinc or sinc² function, or a sech² function.

FIG. 8 also shows a measured spectrum 806, which is simply an array of N spectral values m₁ . . . m_{N} measured by the spectrometer 110 (e.g., the raw spectral values s₁ . . . s_{N} or photocurrents 118(1) . . . 118(N) of FIG. 1). Each spectral value mᵢ is shown in FIG. 8 as a square and indicates an intensity, power, or similar quantity of light. Although each pixel detector 116(i) detects a range of wavelengths, the measured spectrum 806 is plotted such that each spectral value mᵢ is located at the center wavelength λᵢ^{(c)} of the corresponding pixel detector 116(i). For clarity, FIG. 8 shows only five consecutive spectral values mᵢ₋₂, mᵢ₋₁, mᵢ, mᵢ₊₁, and mᵢ₊₂ measured by five respective pixel detectors 116(i−2), 116(i−1), 116(i), 116(i+1), and 116(i+2). However, the following arguments apply to all of the spectral values m₁ . . . m_{N} and all of the pixel detectors 116(1) . . . 116(N) in the detector array 114.

FIG. 8 shows how light at multiple wavelengths contribute to each spectral value mᵢ. At the wavelength λ₁, the kernel ƒᵢ(λ) has the value ƒᵢ(λ₁) which weights the spectral value p(λ₁). Thus, light at the wavelength λ₁ contributes the term ƒᵢ(λ₁)p(λ₁)Δλ to the spectral value mᵢ, where Δλ is a small range of wavelengths over which the spectral density p(λ₁) is assumed to be constant. More generally, light at the wavelength λⱼ contributes the term ƒᵢ(λⱼ)p(λⱼ)Δλ to the spectral value mᵢ. Since both the spectral density p(λ) and kernel ƒᵢ(λ) are continuous functions of wavelength λ, contributions from all wavelengths to the spectral value mᵢ can be expressed as the integral

$${m}_{i} = \int p ( λ ) {f}_{i} ( λ ) d λ , \qquad ( 1 )$$

where the integral is taken over all wavelengths λ.

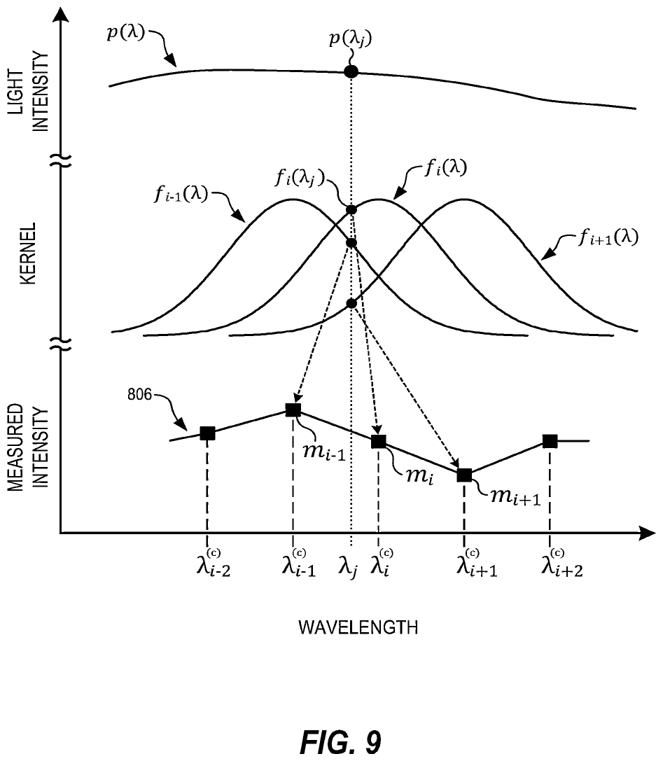

FIG. 9 illustrates how light at the one wavelength λⱼ contributes to several of the spectral values m₁ . . . m_{N}. FIG. 9 is similar to FIG. 8 except that three kernels are shown, specifically the kernel ƒᵢ₋₁(λ) for the pixel detector 118(i−1), the kernel ƒᵢ(λ) for the pixel detector 118(i), and the kernel ƒᵢ₊₁(λ) for the pixel detector 118(i+1). For clarity, FIG. 9 only shows the three consecutive spectral values, mᵢ₋₁, mᵢ, and mᵢ₊₁, measured by the respective pixel detectors 116(i−1), 116(i), and 116(i+1). However, the following arguments apply to all of the spectral values m₁ . . . m_{N} and all of the pixel detectors 116(1) . . . 116(N) in the detector array 114.

At the wavelength λⱼ, the kernel ƒᵢ(λ) has the value ƒᵢ(λⱼ). The resulting contribution to the spectral value mᵢ is ƒᵢ(λ)p(λ)Δλ; this term quantifies how much light in the range Δλ will impinge on the pixel detector 118(i). However, some of the light in this range will also impinge on the pixel detectors 118(i−1) and 118 (i+1). Specifically, at the wavelength λⱼ, the kernel ƒᵢ₋₁(λ) has the value ƒᵢ₋₁(λⱼ). The resulting contribution to the spectral value mᵢ₋₁ is ƒᵢ₋₁(λ)p(λ)Δλ. Similarly, at the wavelength λⱼ, the kernel ƒᵢ₊₁(λ) has the value ƒᵢ₊₁(λⱼ), resulting in a contribution of ƒᵢ₊₁(λ)p(λ)Δλ to the spectral value mᵢ₊₁.

Although not shown in FIG. 9, light at the wavelength λⱼ will also impinge on other pixel detectors 116, thereby contributing to other spectral values (e.g., mᵢ₊₂, mᵢ₊₃, etc.). The amount of light at the wavelength λⱼ that reaches the pixel detector 118(k) is given by the kernel ƒₖ(λ) of the pixel detector 118(k), and therefore decreases exponentially with the square of the wavelength difference λⱼ−λₖ^{(c)}. When |λⱼ−λₖ^{(c)}| is sufficiently large, the value ƒₖ(λⱼ) will be so small that the resulting contribution ƒₖ(λⱼ)p(λⱼ)Δλ is negligible, and therefore can be ignored. Accordingly, the wavelengths that contribute the most to the spectral value m; are usually those that are closest to the center wavelength λᵢ^{(c)} (e.g., within one resolving resolution or standard deviation σ of the center wavelength λᵢ^{(c)}.

FIGS. 8 and 9 also illustrate a method for deconvolving the spectral values m₁ . . . m_{N} into estimates of the spectral density p(λ) that are more accurate than the spectral values m₁ . . . m_{N} themselves. This deconvolving may be implemented with the signal processing circuit 130 of FIG. 1 (e.g., by deconvolving the array 126 of raw spectral values s₁ . . . s_{N} into the array 132 of deconvolved spectral values \tilde{s}₁ . . . \tilde{s}_{N}). With this method, Eqn. 1 can be approximated as:

$${m}_{i} = {\sum }_{k = 1}^{R} {f}_{i} ( {λ}_{k}^{( r )} ) p ( {λ}_{k}^{( r )} ) {Δ}_{k} , \qquad ( 2 )$$

wherein λ₁^{(r)} . . . λ_{R}^{(r)} are R reference wavelengths and Δ₁ . . . Δ_{R} are R wavelength ranges. Each wavelength range Δₖ∈{Δ₁ . . . Δ_{R}} includes the corresponding reference wavelength λₖ^{(r)}∈{λ₁^{(r)} . . . λ_{R}^{(r)}}. The wavelength ranges Δ₁ . . . Δ_{R} may be R different values. Alternatively, the wavelength ranges Δ₁ . . . Δ_{R} may have a common value when the reference wavelengths λ₁^{(r)} . . . λ_{R}^{(r)} are uniformly distributed.

To extend Eqn. 2 to all N of the pixel detectors 116 in the detector array 114, we use the following vector notation:

$$m = {[ {m}_{1} , {m}_{2} , … , {m}_{N} ]}^{T} p = {[ p ( {λ}_{1}^{( r )} ) {Δ}_{1} , p ( {λ}_{2}^{( r )} ) {Δ}_{2} , … , p ( {λ}_{R}^{( r )} ) {Δ}_{R} ]}^{T} {f}_{i} = [ {f}_{i} ( {λ}_{1}^{( r )} ) , {f}_{i} ( {λ}_{2}^{( r )} ) , … , {f}_{i} ( {λ}_{R}^{( r )} ) ] F = {[ {f}_{1}^{T} , {f}_{2}^{T} , … , {f}_{N}^{T} ]}^{T} . \qquad ( 3 )$$

In Eqn. 3, F is a kernel matrix whose rows correspond to the N pixel detectors 116 and whose columns correspond to the R reference wavelengths λ₁^{(r)} . . . λ_{R}^{(r)}. Each element Fᵢⱼ of the kernel matrix F gives the value ƒᵢ(λⱼ^{(r)}), The extension to Eqn. 2 can then be expressed as

$$m = Fp . \qquad ( 4 )$$

The goal is to find a solution \hat{p} given m and F. If the kernel matrix F is well-conditioned, then the pseudo-inverse F⁺ of the matrix F can be calculated. The solution \hat{p} is then given by

$$\hat{p} = {F}^{+} m . \qquad ( 5 )$$

If F is one-to-one, then

$${F}^{+} = {( {F}^{T} F )}^{- 1} {F}^{T} . \qquad ( 6 )$$

However, when F is ill-conditioned, the pseudo-inverse F⁺ will likely not be calculable. In this case, an alternative technique to find the solution \hat{p} is to use least-squares regression to solve

$$\hat{p} = arg min {‖ m - Fp ‖}_{2}^{2} . \qquad ( 7 )$$

by varying p.

Least-squares regression may still not give correct solutions when F is ill-conditioned. In this case, one or more types of regularization can be used. One such type of regularization is Tikhonov regularization. In this case, Eqn. 7 is modified by adding a regularization term ∥Γp∥₂², where Γ is a Tikhonov matrix. The resulting Tikhonov-regularized least-squares regression is given by

$$\hat{p} = arg min {‖ m - Fp ‖}_{2}^{2} + {‖ Γ p ‖}_{2}^{2} . \qquad ( 8 )$$

Many choices for the Tikhonov matrix Γ are known in the art, any of which may be used. One choice is Γ=αU, where α∈ [symbol] ₊ and I is the identity matrix of size R. In this case, the regularization term penalizes large elements of p. A larger value of α produces stronger regularization while a smaller value of a produces weaker regularization, with α=0 leading to the unregularized least-squares regression of Eqn. 7. Another choice for the Tikhonov matrix Γ is Γ=I−αₖIₖ, where αₖ is a constant and Iₖ is the shift matrix with nonzero elements along the k^{th} superdiagonal or subdiagonal. This choice of Γ creates a finite difference matrix that penalizes large values of the k^{th} derivative of p, leading to a smoother solution. In one embodiments, only the first and second superdiagonals are non-zero (i.e., αₖ=0 for all k≥ 3).

The above examples of Γ can be added together to obtain a generalized Tikhonov matrix Γ=α₀I−Σₖ₌₁^{R}αₖIₖ. Tikhonov regularization results in a closed-form solution, just like unregularized least-squares regression. The solution is given by

$$\hat{p} = {F}^{+} m . \qquad ( 9 )$$

When F is one-to-one,

$${F}^{+} = {( {F}^{T} F + {Γ}^{T} Γ )}^{- 1} {F}^{T} . \qquad ( 10 )$$

In embodiments, the center wavelengths λ₁^{(c)} . . . λ_{N}^{(c)} form an ordered sequence and the reference wavelengths λ₁^{(r)} . . . λ_{R}^{(r)} form an ordered sequence. Note that there is no requirement that any of the center wavelengths λ₁^{(c)} . . . λ_{N}^{(C)} equal any of the reference wavelengths λ₁^{(r)} . . . λ_{R}^{(r)}. By comparison, many prior-art deconvolution techniques only consider the case where R=N and λᵢ^{(c)}=λᵢ^{(r)} for all i. Thus, the deconvolution described herein not only “sharpens” the features of the measured spectrum 806, much like prior-art deconvolution techniques, but it also allows each component \hat{p}ᵢ of the solution \hat{p} to be associated with a wavelength that is different from any of the center wavelengths λ₁^{(c)} . . . λ_{N}^{(c)}. This type of deconvolution, in which at least one of the reference wavelengths λ₁^{(r)} . . . λ_{R}^{(r)} is different from all of the center wavelengths λ₁^{(c)} . . . λ_{N}^{(r)}, is referred to herein as wavelength-shifting deconvolution.

Wavelength-shifting deconvolution advantageously accounts for the fact that different spectrometers, even of the same type, will likely have different sets of center wavelengths λ₁^{(c)} . . . λ_{N}^{(c)}. These differences typically arise from manufacturing variations (e.g., tolerances of component fabrication and assembly). However, the machine-learning model 140 will have been trained with training data associated with only the reference wavelengths λ₁^{(r)} . . . λ_{R}^{(r)}, which are likely different from the center wavelengths λ₁^{(c)} . . . λ_{N}^{(c)} of the spectrometer at hand. Wavelength-shifting deconvolution resolves these differences, thereby ensuring that every deconvolved spectral value \tilde{s}ᵢ that is fed into the machine-learning model 140 is associated with the reference wavelength λᵢ^{(r)} that the machine-learning model 140 assumes is associated with the deconvolved spectral value \tilde{s}ᵢ. This shifting of wavelengths increases the accuracy of the indication 142 outputted by the machine-learning model 140.

Since the number R of reference wavelengths λ₁^{(r)} . . . λ_{R}^{(r)} may be different than the number N of center wavelengths λ₁^{(c)} . . . λ_{N}^{(c)}, wavelength-shifting deconvolution also allows the machine-learning model 140 to be used with spectrometers having different numbers of pixel detectors. Specifically, wavelength-shifting deconvolution can be used to transform N raw spectral values into R deconvolved spectral values. For example, consider a first spectrometer with a first detector array having N₁=2048 pixel detectors, a second spectrometer with a second detector array having N₂=3648 pixel detectors, and a machine-learning model having R=2500 inputs (e.g., input nodes of an artificial neural network). Here, a first kernel matrix F₁ can be constructed to transform the N₁=2048 spectral values m₁ . . . m₂₀₄₈ measured with the first detector array into R=2500 wavelength-shifted-and-deconvolved spectral values \tilde{s}₁ . . . \tilde{s}₂₅₀₀ that are fed into the machine-learning model 140. Similarly, a second kernel matrix F₂ can be constructed to transform the N₂=3648 spectral values m₁ . . . m₃₆₄₈ measured with the second detector array into another R=2500 wavelength-shifted-and-deconvolved spectral values \tilde{s}₁ . . . \tilde{s}₂₅₀₀ that are fed into the machine-learning model 140. In both cases, the same machine-learning model 140 is used. While this example shows that R may be greater than or less than N, alternatively R equals N. Furthermore, when R equals N, it is not necessary that each of the reference wavelengths λ₁^{(r)} . . . λ_{R}^{(r)} equals a corresponding one of the center wavelengths λ₁^{(c)} . . . λ_{N}^{(c)} (i.e., λ₁^{(c)}=λ₁^{(r)}, λ₂^{(c)}=λ₂^{(r)}, etc.). Rather, wavelength-shifting deconvolution can be used without changing the numbers of spectral values (in this case, the kernel matrix F will be a square matrix).

Wavelength-shifting deconvolution also helps manufacturing and technical support by promoting modularity of any of the systems described herein. For example, during production, all spectrometers can be tested using the same machine-learning model 140. As an example, consider a system in the field that has a broken spectrometer. This broken spectrometer can be easily replaced with a new spectrometer that likely has a different wavelength calibration (i.e., different center wavelengths λ₁^{(c)} . . . λ_{N}^{(c)}). The spectrometer may include a memory in which it stores its own center wavelengths λ₁^{(c)} . . . λ_{N}^{(c)}. When the new spectrometer is first connected to the system, the signal processing circuit 130 can retrieve the center wavelengths λ₁^{(c)} . . . λ_{N}^{(c)} from the memory. The signal processing circuit 130 may then use these center wavelengths λ₁^{(c)} . . . λ_{N}^{(c)} to construct a new kernel matrix F. Alternatively, the kernel matrix F may be stored in the spectrometer's memory, in which case the signal processing circuit 130 may retrieve the kernel matrix F from the spectrometer's memory without having to perform any matrix construction. In any case, the machine-learning model 140 need not be modified at all.

Using the same machine-learning model 140 with all units is also advantageous because training data may be expensive and difficult to obtain. Accordingly, it is easier, faster, and less expensive to maintain a single “master” model that is updated with new training data and then pushed to all deployed units (e.g., via the internet). Alternatively, when the machine-learning model 140 is deployed on a remote computer server that communicates with field-deployed units, the internal architecture of the machine-learning model 140 (i.e., the architecture other than the number of inputs and the number of outputs) can be modified without changing how the field-deployed units communicate with the remote computer server.

### Constructing the Kernel Matrix F

Constructing the kernel matrix F for a given spectrometer begins with knowledge of the center wavelengths λ₁^{(c)} . . . λ_{R}^{(c)} and reference wavelengths λ₁^{(r)} . . . λ_{R}^{(r)}. The reference wavelengths λ₁^{(r)} . . . λ_{R}^{(r)} may be selected based on the signature of one or more constituents in the fluid 108. Accordingly, some of the present method embodiments include the step of selecting the reference wavelengths λ₁^{(r)} . . . λ_{R}^{(r)}.

The center wavelengths λ₁^{(c)} . . . λ_{R}^{(c)} may be simply measured with the spectrometer at hand. Any determination of the center wavelengths λ₁^{(c)} . . . λ_{R}^{(c)} is referred to as wavelength calibration. As an example of wavelength calibration, consider a tunable source of monochromatic light (e.g., a tunable laser or broadband light source filtered by a monochromator) whose wavelength can be precisely and accurately measured (e.g., with a wavemeter). While this monochromatic light is coupled into the spectrometer 110, the photocurrent 118(i) outputted by the pixel detector 116(i) can be measured at several different wavelengths where the photocurrent 118(i) is maximum. These measurements will form a Gaussian profile (or similar “peaked” distribution), as described above. The measurements may be fit (e.g., nonlinear regression) to a best-fit function to obtain best-fit values for the center wavelength, width (or resolving resolution), and amplitude of the Gaussian profile. This best-fit function estimates the sensitivity function ƒᵢ(λ) of the pixel detector 116(i). The process may be repeated to obtain an estimate of the sensitivity function ƒᵢ(λ) for all N of the pixel detectors 116(1) . . . 116(N).

Since measuring the sensitivity function ƒᵢ(λ) of all of the pixel detectors 116 may be time-consuming, certain assumptions may be made to speed up the process. For example, some of the center wavelengths λ₁^{(c)} . . . λ_{R}^{(c)} may be determined by interpolation. For example, when the sensitivity function ƒ₁(λ) of the first pixel detector 116(1) is measured to obtain the first center wavelength λᵢ^{(c)} and the sensitivity function ƒ_{N}(λ) of the last pixel detector 116(N) is measured to obtain the last center wavelength 1, then all of the intervening center wavelengths λ₂^{(c)} . . . λ_{N-1}^{(c)} may be estimated by linear interpolation assuming that these intervening center wavelengths are uniformly distributed. The resolving resolutions (i.e., Gaussian width) of the pixel detectors 116 may be similarly obtained by linearly interpolating the best-fit resolving resolutions of the first pixel detector 116(1) and last pixel detector 116(N). Alternatively, it may be simply assumed that all of the pixel detectors 116 have the same resolving resolution.

Many manufacturers of spectrometers perform wavelength calibration before shipping units. To make the resulting center wavelengths λ₁^{(c)} . . . λ_{R}^{(c)} available to the customer or user, they may be stored in a memory that is physically co-located with the spectrometer. Alternatively, the center wavelengths λ₁^{(c)} . . . λ_{R}^{(c)} may be printed on a sheet of paper that is shipped with the unit, provided electronically (e.g., on a memory stick), or made available for download from a remote server. In any case, when the manufacturer (or another third party) performs wavelength calibration and provides the resulting center wavelengths λ₁^{(c)} . . . λ_{R}^{(c)}, there is usually no need for the user to repeat this process. Accordingly, some of the present method embodiments may be used to construct a kernel matrix F without performing any type of calibration measurement with the spectrometer. In this case, the sensitivity function ƒᵢ(λ) for the pixel detector 116(i) can be assumed to have a Gaussian form (or similar peaked profile) centered at the provided value of the center wavelength λᵢ^{(c)}. The width of the Gaussian, or resolving resolution of the pixel detector 116(i), may be similarly assumed to have a value provided by the manufacturer (e.g., on a specification sheet or test-data sheet) or interpolated therefrom.

The kernel matrix F has N×R elements denoted Fᵢⱼ, where i indexes the pixel detectors 116 (i.e., i runs from 1 to the number N of the pixel detectors 116) and j indexes the reference wavelengths (i.e., j runs from 1 to the number R of the reference wavelengths a λ₁^{(r)} . . . λ_{R}^{(r)}). The element Fᵢⱼ has the value Fᵢⱼ=ƒᵢ(λⱼ^{(r)}). Thus, once the sensitivity functions are known for all of the pixel detectors 116, the kernel matrix F can be populated by evaluating each sensitivity function ƒᵢ at each of the R reference wavelengths λ₁^{(r)} . . . λ_{R}^{(r)}.

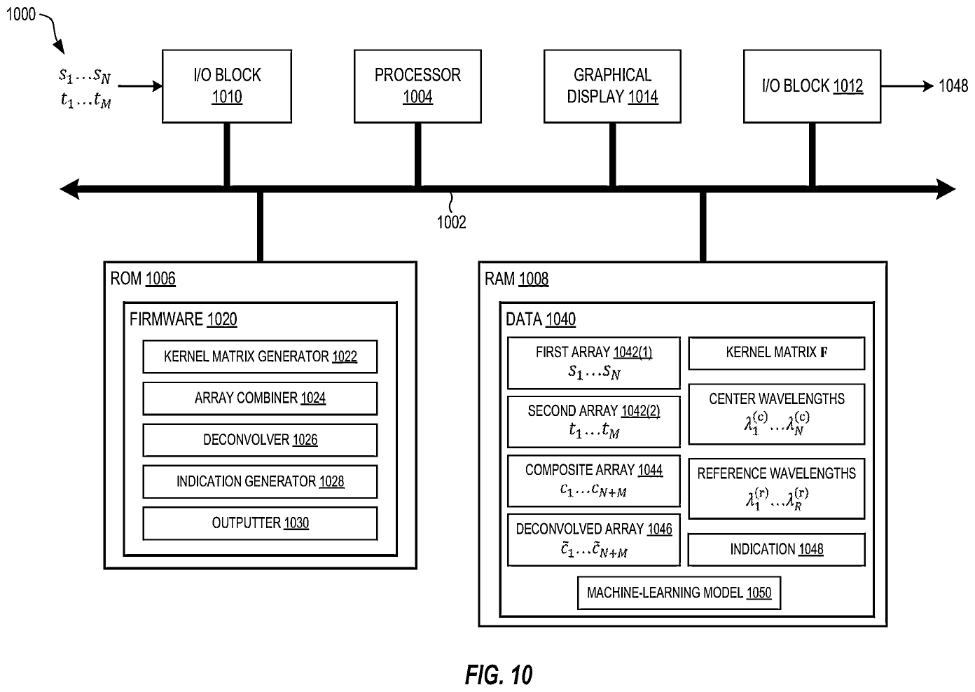

FIG. 10 is a functional diagram of a computational system 1000 that is one example of the signal processing circuit 130 of FIG. 1. The computational system 1000 may be implemented, for example, as an embedded system co-located with other components of any system embodiment described herein (e.g., the system 100 of FIG. 1, the system 200 of FIG. 2, etc.). The computational system 1000 includes a read-only memory (ROM) 1006 and a random-access memory (RAM) 1008 that communicate with a processor 1004 over a system bus 1002. In some embodiments, the computational system 1000 also includes a graphical display 1014 for visually displaying information to a user, receiving input from the user, or both. Alternatively, the computational system 1000 may include a display adapter for use with a graphical display provided by a third party.

The computational system 1000 also includes an input/output (I/O) block 1010 that interfaces with one or more spectrometers to receive an array of raw spectral values. For example, the I/O block 1010 may interface with the spectrometers 210(1) and 210(2) of FIG. 2 to receive the first array 226(1) of raw spectral values s₁ . . . s_{N} and the second array 226(2) of raw spectral values t₁ . . . t_{M}. The computational system 1000 also includes an I/O block 1012 through which it may communicate with a peripheral device or remote computer system (e.g., hard drive, USB port, memory card, network connector, etc.). The I/O blocks 1010 and 1012 are also connected to the system bus 1002, and therefore can communicate with the processor 1004, store data in the RAM 1008, and retrieve data from the RAM 1008.

The processor 1004 may be any type of circuit capable of performing logic, control, and input/output operations. For example, the processor 1004 may include one or more of a microprocessor with one or more central processing unit (CPU) cores, a graphics processing unit (GPU), a digital signal processor (DSP), a field-programmable gate array (FPGA), a system-on-chip (SoC), and a microcontroller unit (MCU). The processor 1004 may also include a memory controller, bus controller, one or more co-processors, and/or other components that manage data flow between the processor 1004 and other components communicably coupled to the system bus 1002. The processor 1004 may be implemented as a single integrated circuit (IC), or as a plurality of ICs. In some embodiments, one or more of the processor 1004, ROM 1006, RAM 1008, I/O block 1010, and I/O block 1012 are implemented as a single IC. The processor 1004 may use a complex instruction set computing (CISC) architecture, or a reduced instruction set computing (RISC) architecture.

The ROM 1006 stores machine-readable instructions (shown as firmware 1020 in FIG. 10) that, when executed by the processor 1004, control the computational system 1000 to implement the functionality described herein. In FIG. 10, the firmware 1020 includes a kernel matrix generator 1022 that constructs and populates the kernel matrix F. The firmware 1020 also includes an array combiner 1024 merges the arrays 226(1) and 226(2) into the composite array 330, thereby implementing the block 302 of the method 300. The firmware 1020 also includes a deconvolver 1026 that deconvolves the composite array 330 to generate the deconvolved array 340, thereby implementing the block 304 of the method 300. The firmware 1020 also includes an indication generator 1028 that feeds the deconvolved array 340 into the machine-learning machine 140 to obtain the indication 142. The firmware 1020 also includes an outputter 1030 that outputs one or more of the indication 142 (e.g., by displaying the indication 142 on the graphical display 1014 or transmitting the indication 142, via the I/O block 1012, to an external computer), the first array 1042(1), the second array 1042(2), the composite array 1044, the deconvolved array 1046 (e.g., by plotting the deconvolved array 1046 on the graphical display 1014), and other data 1040. The ROM 1006 may store additional machine-readable instructions (e.g., operating system instructions, I/O control instructions, etc.) or data without departing from the scope hereof. The ROM 1006 may be implemented using non-volatile memory, such as flash memory, NVRAM, FRAM, MRAM, EEPROM, EPROM, or any combination thereof.

The RAM 1008 stores data 1040 used by the processor 1004 when executing the firmware 1020. In FIG. 10, the data 1040 includes a first array 1042(1) of raw spectral values (e.g., the first array 226(1) of raw spectral values s₁ . . . s_{N} in FIG. 2), a second array 1042(2) of raw spectral values (e.g., the second array 226(2) of raw spectral values t₁ . . . t_{M} in FIG. 2), a composite array 1044 (e.g., the composite array 330 of raw spectral values c₁ . . . c_{N+M} in FIG. 3), a deconvolved array 1046 (e.g., the deconvolved array 340 of deconvolved spectral values \tilde{c}₁ . . . \tilde{c}_{N+M} in FIG. 3), a kernel matrix F (e.g., see Eqn. 3), center wavelengths λ₁^{(c)} . . . λ_{N}^{(c)}, reference wavelengths λ₁^{(r)} . . . λ_{R}^{(r)}, an indication 1048 (e.g., the indication 142 in FIG. 1), and a trained machine-learning model 1050 (e.g., the machine-learning model 140 of FIG. 1). However, the RAM 1008 may store additional data 1040 without departing from the scope hereof.

The RAM 1008 may be implemented using volatile memory, such as DRAM, SRAM, or a combination thereof. Since the kernel matrix F, center wavelengths λ₁^{(c)} . . . λ_{R}^{(c)}, reference wavelengths λ₁^{(r)} . . . λ_{R}^{(r)}, and machine-learning model 1050 are not expected to change often, they may alternatively be stored in non-volatile memory (e.g., the ROM 1006), thereby ensuring that the computational system 1000 retains this information during power loss.

Each of the I/O blocks 1010 and 1012 may implement a communication protocol For example, each of the I/O blocks 1010 and 1012 may be a serial communication interface (e.g., RS-232, RS-422, RS-485, etc.), a parallel communication interface (e.g., GPIB, PCI, SCSI, etc.), a synchronous serial communication interface (e.g., I2C, SPI, SSC, etc.), a universal serial bus (USB) interface, a multi-media card interface (e.g., SD card, Compact Flash, etc.), a wired network interface (e.g., Ethernet, Infiniband, Fibre Channel, etc.), a wireless network interface (e.g., WiFi, Bluetooth, BLE, ZigBee, ANT, etc.), a cellular network interface (e.g., 3G, 4G, 5G, LTE), an optical network interface (e.g., SONET, SDH, IrDA, etc.), and a fieldbus interface. In some embodiments, the computational system 1000 uses a separate I/O block for each spectrometer. Thus, in these embodiments the computational system 1000 may include an additional I/O block such that each of the spectrometers 210(1) and 210(2) communicates with the computational system 1000 via a dedicated I/O block.

Although not shown in FIG. 10, the computational system 1000 may include additional I/O functionality, as needed, for communicating with additional peripheral devices. For example, the computational system 1000 may include one or more additional network interfaces (e.g., an Ethernet port or WiFi adapter) to receive the firmware 1020 for the ROM 1006, update the firmware 1020, update the machine-learning model 1050, or feed the deconvolved array 1046 to a machine-learning model that is executed on a remote computer server. Similarly, the one or more additional network interfaces may be used to obtain one or more of: the center wavelengths λ₁^{(c)} . . . λ_{N}^{(c)}, the reference wavelengths λ₁^{(r)} . . . λ_{R}^{(r)}, and the machine-learning model 1050.

The kernel matrix F may be stored in the RAM 1008 (or the ROM 1006) as a two-dimensional array. Each cell, or element, Fᵢⱼ of the kernel matrix F is indexed by a first index value i of a plurality of first index values (e.g., 1 . . . N) and a second index value j of a plurality of second index values (e.g., 1 . . . M). The first index value i uniquely identifies one of the pixel detectors 216 and 218. The second index value j uniquely identifies one of the reference wavelengths λ₁^{(r)} . . . λ_{R}^{(r)}. The cell Fᵢⱼ stores the spectral response ƒᵢ(λⱼ^{(r)}) of the pixel detector 116(i) identified by the first index value i, where the spectral response ƒᵢ(λⱼ^{(r)}) is evaluated at the reference wavelength λⱼ^{(r)} identified by the second index value j.

The deconvolver 1026 may implement the wavelength-shifting deconvolution described above, or another deconvolution technique known in the art. The deconvolver 1026 may implement least-squares regression, either with or without regularization. When least-squares regression is implemented with Tikhonov regularization, the RAM 1008 or ROM 1006 may additional store a Tikhonov matrix Γ (e.g., see Eqn. 8).

In some embodiments, the processor 1004 does not execute machine-readable instructions (e.g., an FPGA) to implement the functionality described here. Rather, the processor c1004 is pre-programmed to perform tasks and therefore acts like a hard-wired circuit. Accordingly, in these embodiments the functionality is implemented only in hardware and the firmware 1020 may be excluded. In other embodiments, the functionality is implemented only in software. In yet other embodiments, this functionality is implemented as a combination of hardware and software.

While FIG. 10 shows the computational system 1000 with one system bus 1002, the computational system 1000 may be implemented with a different type of architecture without departing from the scope hereof. For example, when the firmware 1020 and data 1040 are stored in separate memories, as shown in FIG. 10, the firmware 1020 and data 1040 may be communicated to/from the processor 1004 using separate buses. In this case, the firmware 1020 and data 1040 may be stored in separate memory spaces, thereby implementing a Harvard architecture. Alternatively, the processor 1004 may include one or more layers of cache so that the computational system 1000 implements a modified Harvard architecture using only the one system bus 1002. In some embodiments, the firmware 1020 is stored as an application in secondary storage (e.g., a hard drive), and loaded into the RAM 1008 upon powering on (i.e., boot up). In this case, the application and the data 1040 share the same memory space, thereby implementing a von Neumann architecture.

### ADDITIONAL EMBODIMENTS

Medical centers (e.g., doctor's offices, urgent care centers, pharmacies, etc.) often have long wait times and high costs due to their dependence on highly skilled and highly paid medical professionals. These professionals, though effective and largely often legally required, create a number of bottlenecks. The present embodiments include Pattern Computer's ProSpectral system, which can reduce the number and severity of these bottlenecks by automating a portion of medical services. The ProSpectral system can theoretically diagnose many diseases using as little as two drops of liquid from a person (e.g., saliva). Due to its speed and case-of-use, several versions of the ProSpectral system can be used as a self-service diagnosis and pharmacy station. This station, which may be constructed as a booth, allows a person who believes they may be unwell to receive a diagnosis and treatment all without waiting for a face-to-face visit with a medical professional. The use of a self-service station also reduces opportunities for contagious disease to spread to other medical professionals and patients.

As an example of how the ProSpectral system could be used as a self-service station, consider a person who thinks they may have the flu. Under conventional prior-art models for providing medical care, the person may go to an urgent care facility to get diagnosed and, if positive, get an antiviral treatment. The patient may have to wait (e.g., 30-60 minutes, or more) before being seen, at which point a medical professional needs to perform a nasal swab, wait 10-20 minutes for the result, and then write and fill a prescription or give the patient a shot. The entire process can easily last an hour, if not more.

In an alternative scenario, the person would, after arriving at the urgent care facility, wait for an available ProSpectral booth. Once in the booth, the person would identify himself or herself. For example, the patient may present a form of identification to be scanned in the booth (e.g., a government ID card, insurance card, etc.) or log-in to the system with pre-determined credentials (e.g., username and password). The person would put two drops of saliva into a cuvette. This task could be performed, for example, by dispensing the cuvette into a receptacle. As another example, a straw is dispensed to fill the cuvette, which is held by the booth and exposed through a small opening. The sample would then be scanned. A diagnosis may then be presented or the patient may be referred to a medical professional (e.g., if the result is inconclusive). In the case the patient is positively diagnosed (e.g., with the flu), the booth can offer a prescription (e.g., antivirals). This may legally require the intervention of a medical professional which can be performed remotely through with booth-integrated video screen, camera, speakers, and microphone. At that point, the person leaves the booth and the medical center. The booth could lock itself and self-clean by means of antiseptic spray and/or exchanging the air in the booth. At that point, the booth is sterilized and ready to receive another person.

To reduce the wait time, multiple booths can be installed at a medical facility. To reduce the cost of more booths, the ProSpectral system may be configured with several booths that interface with a single sensor (e.g., the system 100 of FIG. 1, the system 200 of FIG. 2, the system 700 of FIG. 7, etc.) that is operated in a multiplex fashion. Each booth may be connected to the sensor with a fiber optic cable to transmit the sample reading to the sensor for processing. Each booth may also be in communication with the sensor (e.g., wirelessly or wired) to send and receive digital information (e.g., the indication 142 of FIG. 1).

The ProSpectral system may also be used in medical research and clinical trials. Examples include scientific discovery, treatment monitoring, screening for specific diseases, dose response in drugs administered to patients, screening for adverse reactions, and population monitoring for epidemiology. Many of these uses advantageously improve predictions (e.g., accuracy, timeliness, etc.) of drug efficacy, efficiency, sensitivity, toxicity, and side effects. Many of these uses also improve identification of patients who are well-suited-or unsuited-for trial participation and/or eventual treatment. In particular, the ProSpectral system could also be used to facilitate early intervention, i.e., identify early warning signs of possible adverse effects, especially for drugs with a high percentage of side effects (e.g., 3-5%).

Many prior-art instruments and techniques are used to plan, execute, and improve clinical trials. These can include any number of tests to evaluate treatment impact and subject health. Tests vary greatly in nature (e.g., physical\blood pressure, chemical reagent\blood panel, cultures\bacterial infection, DNA sequencing\hereditary conditions). A high percentage of biomedical tests must use well-established sample types (e.g., blood, urine, tissue) and biochemistry e.g., (reagents). Most of the ensuing bioinformatic/biostatistical analysis relies on well-known mathematics that can only validate hypotheses about patterns and correlations, but not inherently discover and explain them.

The present embodiments help overcome many of these limitations. One example of such an improvement is the ability to substitute multiple individual tests with one multiplexed of multi-purpose test. The disadvantage of being constrained to use individual tests, taken as a whole, includes their invasive nature, time-to-results, cost, single-purpose, consumption of supplies and reagents, infrastructure requirements, and required medical licensure. By helping to overcome these disadvantages, the present embodiments can reduce time and cost of clinical trials.

Many of the present embodiments have several advantageous features, such as:
Three-second time-to-results enables vastly faster diagnosis, boosting patient throughput and satisfaction;
A balanced accuracy of 98.5% matches that of PCR tests;
No reagents are required—no toxic waste for facility to handle and dispose of;
As few as two drops of saliva required—no invasive, unpleasant nasopharyngeal swabs;
Small and portable (shoe box-sized and <3.5 kg)—ideal for mobile testing;
Can process over 240 samples per hour;
Simple operation, training, and maintenance-no medical licensure required;
Detecting new diseases and variants only requires a remote software update; and
Less expensive than other tests requiring reagents and complex equipment.

Pattern Computer's Pattern Discovery Engine (PDE), as implemented with many of the present embodiments, can discover patterns in biomarker absorption signatures that reliably correlate with infection by a specific disease, and create an analytical model to test for that disease. It can also identify patterns in data that are eventually determined to be signatures of other events and states (e.g., toxicity, side-effects).

The PDE can process the data that ProSpectral reads from a saliva sample in conjunction with external data—often only obtained in reaction to an issue—about the subject's health (e.g., infection with a given disease, measures of toxicity, measures of drug effectiveness, co-morbidities, allergy/side-effect susceptibility, etc.). This enables it to identify patterns it discovers in the spectrophotometric data as signatures of these other factors. Once such a correlation is established, ProSpectral can report detection of those factors independently and much earlier than often otherwise realized. This improves the safety, planning, execution, timeline, and cost of clinical trials and studies. Once a baseline saliva scan is collected for subjects prior to trial/study start, longitudinal testing (repeated testing during the study) enables the creation of models that can:
Identify which candidate subjects are well-suited-or unsuited-for trial participation;
Monitor for changes in saliva scan data that correlate with current or future changes in effectiveness/toxicity/side effects, and proactive adjustment (e.g., of dosage);
Predict which subjects may be more vulnerable to toxicity or side effects;
Predict treatment effectivity for individual subjects; and
Identify changes that may predict issues usually only apparent after later/higher doses.

The technology that the ProSpectral system uses is fundamentally different from most current relevant over-the-counter technologies for disease detection. It detects the host metabolic response, not the disease itself (e.g., virus). Distinct disease states leave specific traces (signatures) in the molecular composition of these biomarkers. While biosamples have the same chemical groups, bond structures and chemical species abundances may differ widely. The ProSpectral system can profile them precisely, using fine spectral resolution and a signal sensitive to chemical bond length and angles of 0.01 Å and 1°, respectively.

Importantly, it is by design a multiplex device intended to detect additional diseases and variants, and able to analyze other bodily fluids (i.e., blood and urine) as needs evolve. Updating the device to detect for additional diseases or new variants only requires a software update that can be performed remotely.

### Combinations of Features

Features described above as well as those claimed below may be combined in various ways without departing from the scope hereof. The following examples illustrate possible, non-limiting combinations of features and embodiments described above. It should be clear that other changes and modifications may be made to the present embodiments without departing from the spirit and scope of this invention:

(A1) A system includes a light source operable to emit broadband light and a cassette holder that is shaped to receive a test cassette that confines a fluid. The cassette holder is positioned such that the broadband light, when emitted by the light source, passes through the test cassette. The system also includes a spectrometer that, in response to receiving the broadband light after passing through the test cassette, disperses the broadband light onto a plurality of pixel detectors of a detector array. The system also includes a digitizer that is electrically connected to the detector array and operable to read an array of raw spectral values from the detector array. The system also includes a signal processing circuit programmed to deconvolve, based on a spectral response of each of the plurality of pixel detectors, the array of raw spectral values to generate an array of deconvolved spectral values. The signal processing circuit is also programmed to feed the array of deconvolved spectral values into a trained machine-learning model that processes the array of deconvolved spectral values to obtain an indication of the presence of one or more constituents in the fluid. The signal processing circuit is also programmed to output the indication.

(A2) In the system denoted (A1), the spectrometer operates over a wavelength range and the broadband light spans the wavelength range.

(A3) In the system denoted (A2), the wavelength range includes at least a portion of the visible region of the electromagnetic spectrum.

(A4) In either of the systems denoted (A2) and (A3), the wavelength range is 350-750 nm.

(A5) In any of the systems denoted (A2) to (A4), the wavelength range includes at least a portion of the near-infrared region of the electromagnetic spectrum.

(A6) In any of the systems denoted (A2) to (A5), the wavelength range is 900-1700 nm.

(A7) In any of the systems denoted (A1) to (A6), the spectrometer has a resolving resolution in the range of 0.2-3 nm, inclusive.

(A8) In any of the systems denoted (A1) to (A7), the spectrometer is a spectrophotometer.

(A9) In any of the systems denoted (A1) to (A8), the cassette holder includes a cuvette holder and the test cassette includes a cuvette.

(A10) In the system denoted (A9), the cuvette includes glass, plastic, or a combination thereof.

(A11) In any of the systems denoted (A1) to (A10), the fluid includes human saliva.

(A12) In any of the systems denoted (A1) to (A11), the fluid includes a biological fluid obtained from a human or animal.

(A13) In any of the systems denoted (A1) to (A12), the signal processing circuit includes a memory storing a kernel matrix having a plurality of cells. Each cell of the plurality of cells is indexed by a first index value of a plurality of first index values and a second index value of a plurality of second index values. The first index value uniquely identifies one of the plurality of pixel detectors. The second index value uniquely identifies one of a plurality of reference wavelengths. Each cell stores the spectral response of the one of the plurality of pixel detectors identified by the first index value of said each cell, the spectral response being at the one of the plurality of reference wavelengths identified by the second index value of said each cell. The signal processing circuit is further programmed to deconvolve the array of raw spectral values by performing least-squares regression based at least on the array of raw spectral values and the kernel matrix.

(A14) In the system denoted (A13), the signal processing circuit is programmed to perform least-squares regression with Tikhonov regularization.

(A15) In any of the systems denoted (A1) to (A14), the signal processing circuit includes a field-programmable gate array.

(A16) In any of the systems denoted (A1) to (A15), the signal processing circuit includes a processor and a memory communicably coupled to the processor. The signal processing circuit also includes a deconvolution engine implemented as machine-readable instructions that are stored in the memory and, when executed by the processor, control the signal processing circuit to deconvolve the array of raw spectral values to obtain the array of deconvolved spectral values.

(A17) In the system denoted (A16), the memory stores a kernel matrix comprising a plurality of cells. Each cell of the plurality of cells is indexed by a first index value of a plurality of first index values and a second index value of a plurality of second index values. The first index value uniquely identifies one of the plurality of pixel detectors. The second index value uniquely identifies one of a plurality of reference wavelengths. Each cell stores the spectral response of the one of the plurality of pixel detectors identified by the first index value of said each cell, the spectral response being at the one of the plurality of reference wavelengths identified by the second index value of said each cell. The machine-readable instructions that, when executed by the processor, control the signal processing circuit to deconvolve include machine-readable instructions that, when executed by the processor, control the signal-processing circuit to perform least-squares regression based at least on the array of raw spectral values and the kernel matrix.

(A18) In the system denoted (A17), the machine-readable instructions that, when executed by the processor, control the signal processing circuit to perform least-squares regression include machine-readable instructions that, when executed by the processor, control the signal processing circuit to perform least-squares regression with Tikhonov regularization.

(A19) In any of the systems denoted (A1) to (A18), the signal processing circuit is further programmed to feed the array of deconvolved spectral values into the trained machine-learning model by transmitting the array of deconvolved spectral values to an external computer system that feeds the array of deconvolved spectral values into the trained machine-learning model to obtain the indication. The signal processing circuit is further programmed to receive the indication from the external computer system.

(A20) In any of the systems denoted (A1) to (A19), the test cassette is void of any reagent.

(B1) A method includes transmitting broadband light through a fluid confined within a test cassette; dispersing the broadband light, after passing through the test cassette, onto a plurality of pixel detectors forming a detector array; reading an array of raw spectral values from the detector array; deconvolving, based on a spectral response of each of the plurality of pixel detectors, the array of raw spectral values to generate an array of deconvolved spectral values; feeding the array of deconvolved spectral values into a trained machine-learning model that processes the array of deconvolved spectral values to obtain an indication of the presence of one or more constituents in the fluid; and outputting the indication.

(B2) In the method denoted (B1), the method further includes collecting the fluid.

(B3) In the method denoted (B2), said collecting excludes adding any reagent to the fluid.

(B4) In either of the methods denoted (B2) and (B3), said collecting includes inserting at least part of the fluid into the test cassette and placing the test cassette in a cassette holder.

(B5) In any of the methods denoted (B2) to (B4), said collecting includes collecting the fluid from a human patient.

(B6) In the method denoted (B5), said collecting includes collecting saliva from the human patient.

(B7) In either of the methods denoted (B5) and (B6), the method further includes diagnosing, based on the indication, the human patient with a disease.

(B8) In the method denoted (B7), the method further includes providing the human patient with a therapeutic intervention for treating the disease.

(B9) In the method denoted (B8), the therapeutic intervention is a surgical procedure, a non-surgical medical procedure, a prescription for one or more pharmaceutical drugs, or a combination thereof.

(B10) In any of the methods denoted (B1) to (B9), said deconvolving includes performing least-squares regression based at least on the array of raw spectral values and a kernel matrix comprising a plurality of cells; each cell of the plurality of cells is indexed by a first index value of a plurality of first index values and a second index value of a plurality of second index values; the first index value uniquely identifies one of the plurality of pixel detectors; the second index value uniquely identifies one of a plurality of reference wavelengths; and each cell stores the spectral response of the one of the plurality of pixel detectors identified by the first index value of said each cell, the spectral response being at the one of the plurality of reference wavelengths identified by the second index value of said each cell.

(B11) In the method denoted (B10), said performing least-squares regression includes performing least-squares regression with Tikhonov regularization.

(B12) In any of the methods denoted (B1) to (B11), said feeding includes transmitting the array of deconvolved spectral values to an external computer system, wherein the external computer system feeds the array of deconvolved spectral values into the trained machine-learning model. Said feeding also includes receiving the indication from the external computer system.

(C1) A system includes a light source operable to emit broadband light and cassette holder that is shaped to receive a test cassette that confines a fluid. The cassette holder is positioned such that the broadband light, when emitted by the light source, passes through the test cassette. The system also includes a first spectrometer that, in response to receiving a first portion of the broadband light after the broadband light has passed through the test cassette, disperses a first wavelength range of the first portion of the broadband light onto a first plurality of pixel detectors forming a first detector array. The system also includes a second spectrometer that, in response to receiving a second portion of the broadband light after the broadband light has passed through the test cassette, disperses a second wavelength range of the second portion of the broadband light onto a second plurality of pixel detectors forming a second detector array. The system also includes a digitizer that is electrically connected to the first and second detector arrays. The digitizer is operable to read a first array of raw spectral values from the first detector array and read a second array of raw spectral values from the second detector array. The system also includes a signal processing circuit programmed to deconvolve, based on a spectral response of each of the first and second pluralities of pixel detectors, the first and second arrays of raw spectral values to generate an array of deconvolved spectral values; feed the array of deconvolved spectral values into a trained machine-learning model that processes the array of deconvolved spectral values to obtain an indication of the presence of one or more constituents in the fluid; and output the indication.

(C2) In the system denoted (C1), the signal processing circuit is further programmed to (i) merge the first and second arrays of raw spectral values into a composite array of raw spectral values and (ii) deconvolve the composite array of raw spectral values to obtain the array of deconvolved spectral values.

(C3) In the system denoted (C1), the signal processing circuit is further programmed to deconvolve the first array of raw spectral values to generate a first partial array of deconvolved spectral values; deconvolve the second array of raw spectral values to generate a second partial array of deconvolved spectral values; and merge the first and second partial arrays of deconvolved spectral values to obtain the array of deconvolved spectral values.

(C4) In any of the systems denoted (C1) to (C3), the first wavelength range includes at least part of the visible region of the electromagnetic spectrum and the second wavelength range includes at least part of the near-infrared region of the electromagnetic spectrum.

(C5) In the system denoted (C4), the first wavelength range includes 350-700 nm and the second wavelength range includes 900-1700 nm.

(C6) In any of the systems denoted (C1) to (C5), the first and second wavelength ranges partially overlap.

(C7) In any of the systems denoted (C1) to (C5), the first and second wavelength ranges do not overlap.

(C8) In any of the systems denoted (C1) to (C7), the first spectrometer has a first resolving resolution and the second spectrometer has a second resolving resolution different than the first resolving resolution.

(C9) In any of the systems denoted (C1) to (C8), each of the first and second spectrometers includes a spectrophotometer.

(C10) In any of the systems denoted (C1) to (C9), the system further includes an input fiber-optic cable; first and second spectrometer fiber-optic cables; a fiber collimator that couples the broadband light, after passing through the test cassette, into the input fiber-optic cable; and an optical splitter that divides the broadband light exiting the input fiber-optic cable into the first and second portions, couples the first portion into the first spectrometer fiber-optic cable, and couples the second portion into the second spectrometer fiber-optic cable. The first spectrometer fiber-optic cable couples the first portion into the first spectrometer. The second spectrometer fiber-optic cable couples the second portion into the second spectrometer.

(C11) In any of the systems denoted (C1) to (C10), the cassette holder includes a cuvette holder that positions a cuvette such that the fluid, when confined within the cuvette, is illuminated by the broadband light.

(C12) In the system denoted (C11), the cuvette includes glass, plastic, or a combination thereof.

(C13) In any of the systems denoted (C1) to (C12), the fluid includes human saliva.

(C14) In any of the systems denoted (C1) to (C13), the fluid includes a biological fluid obtained from a human or animal.

(C15) In any of the systems denoted (C1) to (C14), the signal processing circuit includes a memory storing a kernel matrix having a plurality of cells. Each cell of the plurality of cells is indexed by a first index value of a plurality of first index values and a second index value of a plurality of second index values. The first index value uniquely identifies one of the first and second pluralities of pixel detectors. The second index value uniquely identifies one of a plurality of reference wavelengths. Each cell stores the spectral response of the one of the first and second pluralities of pixel detectors identified by the first index value of said each cell, the spectral response being at the one of the plurality of reference wavelengths identified by the second index value of said each cell. The signal processing circuit is further programmed to deconvolve the first and second arrays of raw spectral values by performing least-squares regression based at least on the first and second arrays of raw spectral values and the kernel matrix.

(C16) In the system denoted (C15), the signal processing circuit is programmed to perform least-squares regression with Tikhonov regularization.

(C17) In either of the systems denoted (C15) and (C16), at least one of the reference wavelengths lies within the first wavelength range and at least one of the reference wavelengths lies within the second wavelength range.

(C18) In any of the systems denoted (C1) to (C17), the signal processing circuit includes a field-programmable gate array.

(C19) In any of the systems denoted (C1) to (C18), the signal processing circuit includes a processor, a memory communicably coupled to the processor, and a deconvolution engine implemented as machine-readable instructions that are stored in the memory and, when executed by the processor, control the signal processing circuit to deconvolve the first and second arrays of raw spectral values to obtain the array of deconvolved spectral values.

(C20) In the system denoted (C19), the memory stores a kernel matrix having a plurality of cells. Each cell of the plurality of cells is indexed by a first index value of a plurality of first index values and a second index value of a plurality of second index values. The first index value uniquely identifies one of the first and second pluralities of pixel detectors. The second index value uniquely identifies one of a plurality of reference wavelengths. Each cell stores the spectral response of the one of the first and second pluralities of pixel detectors identified by the first index value of said each cell, the spectral response being at the one of the plurality of reference wavelengths identified by the second index value of said each cell. The machine-readable instructions that, when executed by the processor, control the signal processing circuit to deconvolve include machine-readable instructions that, when executed by the processor, control the signal processing circuit to perform least-squares regression based at least on the first and second arrays of raw spectral values and the kernel matrix.

(C21) In the system denoted (C20), the machine-readable instructions that, when executed by the processor, control the signal processing circuit to perform least-squares regression include machine-readable instructions that, when executed by the processor, control the signal processing circuit to perform least-squares regression with Tikhonov regularization.

(C22) In either of the systems denoted (C20) and (C21), at least one of the reference wavelengths lies within the first wavelength range and at least one of the reference wavelengths lies within the second wavelength range.

(C23) In any of the systems denoted (C1) to (C22), the signal processing circuit is further programmed to feed the array of deconvolved spectral values into the trained machine-learning model by transmitting the array of deconvolved spectral values to an external computer system that feeds the array of deconvolved spectral values into the trained machine-learning model to obtain the indication. The signal processing circuit is further programmed to receive the indication from the external computer system.

(D1) A method includes transmitting broadband light through a fluid confined within a test cassette; dispersing a first wavelength range of the broadband light, after passing through the test cassette, onto a first plurality of pixel detectors forming a first detector array; dispersing a second wavelength range of the broadband light, after passing through the test cassette, onto a second plurality of pixel detectors forming a second detector array; reading a first array of raw spectral values from the first detector array; reading a second array of raw spectral values from the second detector array; deconvolving, based on a spectral response of each pixel detector of the first and second pluralities of pixel detectors, the first and second arrays of raw spectral values to generate an array of deconvolved spectral values; feeding the array of deconvolved spectral values into a trained machine-learning model that processes the array of deconvolved spectral values to obtain an indication of the presence of one or more constituents in the fluid; and outputting the indication.

(D2) In the method denoted (D1), the method further includes merging the first and second arrays of raw spectral values into a composite array of raw spectral values. Said deconvolving includes deconvolving the composite array of raw spectral values.

(D3) In the method denoted (D1), said deconvolving includes deconvolving the first array of raw spectral values to obtain a first partial array of deconvolved spectral values and deconvolving the second array of raw spectral values to obtain a second partial array of deconvolved spectral values. The method further includes merging the first and second partial arrays of deconvolved spectral values to obtain the array of deconvolved spectral values.

(D4) In any of the methods denoted (D1) to (D3), the method further includes collecting the fluid.

(D5) In the method denoted (D4), said collecting excludes adding any reagent to the fluid.

(D6) In either of the methods denoted (D4) and (D5), said collecting includes inserting at least part of the fluid into the test cassette and placing the test cassette in a cassette holder.

(D7) In any of the methods denoted (D4) to (D6), said collecting includes collecting the fluid from a human patient.

(D8) In the method denoted (D7), said collecting includes comprises saliva from the human patient.

(D9) In either of the methods denoted (D6) and (D7), the method further includes diagnosing, based on the indication, the human patient with a disease.

(D10) In the method denoted (D9), the method further includes providing the human patient with a therapeutic intervention for treating the disease.

(D11) In the method denoted (D10), the therapeutic intervention being a surgical procedure, a non-surgical medical procedure, a prescription for one or more pharmaceutical drugs, or a combination thereof.

(D12) In any of the methods denoted (D1) to (D11), said deconvolving includes performing least-squares regression based at least on the first and second arrays of raw spectral values and a kernel matrix comprising a plurality of cells; each cell of the plurality of cells is indexed by a first index value of a plurality of first index values and a second index value of a plurality of second index values; the first index value uniquely identifies one of the first and second pluralities of pixel detectors; the second index value uniquely identifies one of a plurality of reference wavelengths; and each cell stores the spectral response of the one of the first and second pluralities of pixel detectors identified by the first index value of said each cell, the spectral response being at the one of the plurality of reference wavelengths identified by the second index value of said each cell.

(D13) In the method denoted (D12), said performing least-squares regression includes performing least-squares regression with Tikhonov regularization.

(D14) In any of the methods denoted (D1) to (D13), said feeding includes transmitting the array of deconvolved spectral values to an external computer system, wherein the external computer system feeds the array of deconvolved spectral values into the trained machine-learning model. Said feeding also includes receiving the indication from the external computer system.

(E1) A system includes a first light source operable to emit a first broadband light beam, a second light source operable to emit a second broadband light beam, and a cassette holder that is shaped to receive a test cassette that confines a fluid. The cassette holder is positioned such that the first and second broadband light beams, when emitted by the respective first and second light sources, pass through the test cassette. The system also includes a first spectrometer that, in response to receiving the first broadband light beam after the first broadband light beam has passed through the test cassette, disperses the first broadband light beam onto a first plurality of pixel detectors forming a first detector array. The system also includes a second spectrometer that, in response to receiving the second broadband light beam after the second broadband light beam has passed through the test cassette, disperses the second broadband light beam onto a second plurality of pixel detectors forming a second detector array. The system also includes a digitizer that is electrically connected to the first and second detector arrays. The digitizer is operable to read a first array of raw spectral values from the first detector array and read a second array of raw spectral values from the second detector array. The system also includes a signal processing circuit programmed to deconvolve, based on a spectral response of each of the first and second pluralities of pixel detectors, the first and second arrays of raw spectral values to obtain an array of deconvolved spectral values; feed the array of deconvolved spectral values into a trained machine-learning model that processes the array of deconvolved spectral values to obtain an indication of the presence of one or more constituents in the fluid; and output the indication.

(F1) A method includes constructing a kernel matrix of a spectrometer. The kernel matrix comprises a plurality of cells. Each cell of the plurality of cells is indexed by a first index value of a plurality of first index values and a second index value of a plurality of second index values. The first index value uniquely identifies one of a plurality of pixel detectors of the spectrometer. The second index value uniquely identifies one of a plurality of reference wavelengths. Said constructing includes, for each pixel detector of the plurality of pixel detectors: constructing a spectral response function based at least on (i) one of a plurality of central wavelengths corresponding to said each pixel detector and (ii) one of a plurality of spectral widths corresponding to said each pixel detector; evaluating the spectral response function at the plurality of reference wavelengths to obtain a pixel-response vector; and inserting the pixel-response vector into the kernel matrix.

(F2) In the method denoted (F1), the method further includes downloading the plurality of reference wavelengths from a memory of the spectrometer.

(F3) In either of the methods denoted (F1) and (F2), the method further includes setting all of the plurality of spectral widths to an identical value.

(F4) In any of the methods denoted (F1) to (F3), said constructing the spectral response function includes constructing a Gaussian function that is centered at said one of the plurality of central wavelengths and has a width based on said one of the plurality of spectral widths.

(F5) In any of the methods denoted (F1) to (F4), the method further includes uploading the kernel matrix to a memory of the spectrometer.

(F6) In any of the methods denoted (F1) to (F5), the spectrometer includes a spectrophotometer.

(F7) In any of the methods denoted (F1) to (F6), the plurality of reference wavelengths are uniformly spaced.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system comprising:
a light source operable to emit broadband light;
a cassette holder that is:
shaped to receive a test cassette that confines a fluid; and
positioned such that the broadband light, when emitted by the light source, passes through the test cassette;
a spectrometer that, in response to receiving the broadband light after passing through the test cassette, disperses the broadband light onto a plurality of pixel detectors of a detector array;
a digitizer that is electrically connected to the detector array and operable to read an array of raw spectral values from the detector array; and
a signal processing circuit programmed to:
deconvolve, based on a spectral response of each of the plurality of pixel detectors, the array of raw spectral values to generate an array of deconvolved spectral values;
feed the array of deconvolved spectral values into a trained machine-learning model that processes the array of deconvolved spectral values to obtain an indication of the presence of one or more constituents in the fluid; and
output the indication.

2. The system of claim 1, wherein:
the spectrometer operates over a wavelength range; and
the broadband light spans the wavelength range.

3. The system of claim 2, the wavelength range comprising at least a portion of the visible region of the electromagnetic spectrum.

4. The system of claim 2, the wavelength range comprising 350-750 nm.

5. The system of claim 2, the wavelength range comprising at least a portion of the near-infrared region of the electromagnetic spectrum.

6. The system of claim 2, the wavelength range comprising 900-1700 nm.

7. The system of claim 1, the spectrometer having a resolving resolution in the range of 0.2-3 nm, inclusive.

8. The system of claim 1, the spectrometer comprising a spectrophotometer.

9. The system of claim 1, the cassette holder comprising a cuvette holder, the test cassette comprising a cuvette.

10. The system of claim 9, the cuvette comprising glass or plastic.

11. The system of claim 1, the fluid comprising human saliva.

12. The system of claim 1, the fluid comprising a biological fluid obtained from a human or animal.

13. The system of claim 1, wherein:
the signal processing circuit comprises a memory storing a kernel matrix comprising a plurality of cells, wherein:
each cell of the plurality of cells is indexed by a first index value of a plurality of first index values and a second index value of a plurality of second index values;
the first index value uniquely identifies one of the plurality of pixel detectors;
the second index value uniquely identifies one of a plurality of reference wavelengths; and
each cell stores the spectral response of the one of the plurality of pixel detectors identified by the first index value of said each cell, the spectral response being at the one of the plurality of reference wavelengths identified by the second index value of said each cell; and
the signal processing circuit is further programmed to deconvolve the array of raw spectral values by performing least-squares regression based at least on the array of raw spectral values and the kernel matrix.

14. The system of claim 13, wherein the signal processing circuit is programmed to perform least-squares regression with Tikhonov regularization.

15. The system of claim 1, the signal processing circuit comprising a field-programmable gate array.

16. The system of claim 1, the signal processing circuit comprising:
a processor;
a memory communicably coupled to the processor; and
a deconvolution engine implemented as machine-readable instructions that are stored in the memory and, when executed by the processor, control the signal processing circuit to deconvolve the array of raw spectral values to obtain the array of deconvolved spectral values.

17. The system of claim 16, wherein:
the memory stores a kernel matrix comprising a plurality of cells, wherein:
each cell of the plurality of cells is indexed by a first index value of a plurality of first index values and a second index value of a plurality of second index values;
the first index value uniquely identifies one of the plurality of pixel detectors;
the second index value uniquely identifies one of a plurality of reference wavelengths; and
each cell stores the spectral response of the one of the plurality of pixel detectors identified by the first index value of said each cell, the spectral response being at the one of the plurality of reference wavelengths identified by the second index value of said each cell; and
the machine-readable instructions that, when executed by the processor, control the signal processing circuit to deconvolve include machine-readable instructions that, when executed by the processor, control the signal-processing circuit to perform least-squares regression based at least on the array of raw spectral values and the kernel matrix.

18. The system of claim 17, wherein the machine-readable instructions that, when executed by the processor, control the signal processing circuit to perform least-squares regression include machine-readable instructions that, when executed by the processor, control the signal processing circuit to perform least-squares regression with Tikhonov regularization.

19. The system of claim 1, wherein the signal processing circuit is further programmed to:
feed the array of deconvolved spectral values into the trained machine-learning model by transmitting the array of deconvolved spectral values to an external computer system that feeds the array of deconvolved spectral values into the trained machine-learning model to obtain the indication; and
receive the indication from the external computer system.

20. The system of claim 1, the test cassette being void of any reagent.

21. A method comprising:
transmitting broadband light through a fluid confined within a test cassette;
dispersing the broadband light, after passing through the test cassette, onto a plurality of pixel detectors forming a detector array;
reading an array of raw spectral values from the detector array;
deconvolving, based on a spectral response of each of the plurality of pixel detectors, the array of raw spectral values to generate an array of deconvolved spectral values;
feeding the array of deconvolved spectral values into a trained machine-learning model that processes the array of deconvolved spectral values to obtain an indication of the presence of one or more constituents in the fluid; and
outputting the indication.

22. The method of claim 21, further comprising collecting the fluid.

23. The method of claim 22, wherein said collecting excludes adding any reagent to the fluid.

24. The method of claim 22, wherein said collecting comprises:
inserting at least part of the fluid into the test cassette; and
placing the test cassette in a cassette holder.

25. The method of claim 22, wherein said collecting comprises collecting the fluid from a human patient.

26. The method of claim 25, wherein said collecting comprises collecting saliva from the human patient.

27. The method of claim 25, further comprising diagnosing, based on the indication, the human patient with a disease.

28. The method of claim 27, further comprising providing the human patient with a therapeutic intervention for treating the disease.

29. The method of claim 28, the therapeutic intervention being a surgical procedure, a non-surgical medical procedure, a prescription for one or more pharmaceutical drugs, or a combination thereof.

30. The method of claim 21, wherein:
said deconvolving comprises performing least-squares regression based at least on the array of raw spectral values and a kernel matrix comprising a plurality of cells;
each cell of the plurality of cells is indexed by a first index value of a plurality of first index values and a second index value of a plurality of second index values;
the first index value uniquely identifies one of the plurality of pixel detectors;
the second index value uniquely identifies one of a plurality of reference wavelengths; and
each cell stores the spectral response of the one of the plurality of pixel detectors identified by the first index value of said each cell, the spectral response being at the one of the plurality of reference wavelengths identified by the second index value of said each cell.

31. The method of claim 30, wherein said performing least-squares regression comprises performing least-squares regression with Tikhonov regularization.

32. The method of claim 21, wherein said feeding comprises:
transmitting the array of deconvolved spectral values to an external computer system, wherein the external computer system feeds the array of deconvolved spectral values into the trained machine-learning model; and
receiving the indication from the external computer system.

33. A system comprising:
a light source operable to emit broadband light;
a cassette holder that is:
shaped to receive a test cassette that confines a fluid; and
positioned such that the broadband light, when emitted by the light source, passes through the test cassette;
a first spectrometer that, in response to receiving a first portion of the broadband light after the broadband light has passed through the test cassette, disperses a first wavelength range of the first portion of the broadband light onto a first plurality of pixel detectors forming a first detector array;
a second spectrometer that, in response to receiving a second portion of the broadband light after the broadband light has passed through the test cassette, disperses a second wavelength range of the second portion of the broadband light onto a second plurality of pixel detectors forming a second detector array;
a digitizer that is electrically connected to the first and second detector arrays, the digitizer being operable to:
read a first array of raw spectral values from the first detector array; and
read a second array of raw spectral values from the second detector array; and
a signal processing circuit programmed to:
deconvolve, based on a spectral response of each of the first and second pluralities of pixel detectors, the first and second arrays of raw spectral values to generate an array of deconvolved spectral values;
feed the array of deconvolved spectral values into a trained machine-learning model that processes the array of deconvolved spectral values to obtain an indication of the presence of one or more constituents in the fluid; and
output the indication.

34. The system of claim 33, wherein the signal processing circuit is further programmed to:
merge the first and second arrays of raw spectral values into a composite array of raw spectral values; and
deconvolve the composite array of raw spectral values to obtain the array of deconvolved spectral values.

35. The system of claim 33, wherein the signal processing circuit is further programmed to:
deconvolve the first array of raw spectral values to generate a first partial array of deconvolved spectral values;
deconvolve the second array of raw spectral values to generate a second partial array of deconvolved spectral values; and
merge the first and second partial arrays of deconvolved spectral values to obtain the array of deconvolved spectral values.

36. The system of claim 33, wherein:
the first wavelength range comprises at least part of the visible region of the electromagnetic spectrum; and
the second wavelength range comprises at least part of the near-infrared region of the electromagnetic spectrum.

37. The system of claim 33, wherein:
the first wavelength range comprises 350-700 nm; and
the second wavelength range comprises 900-1700 nm.

38. The system of claim 33, wherein the first and second wavelength ranges partially overlap.

39. The system of claim 33, wherein the first and second wavelength ranges do not overlap.

40. The system of claim 33, wherein:
the first spectrometer has a first resolving resolution; and
the second spectrometer has a second resolving resolution different than the first resolving resolution.

41. The system of claim 33, each of the first and second spectrometers comprising a spectrophotometer.

42. The system of claim 33, further comprising:
an input fiber-optic cable;
first and second spectrometer fiber-optic cables;
a fiber collimator that couples the broadband light, after passing through the test cassette, into the input fiber-optic cable; and
an optical splitter that:
divides the broadband light exiting the input fiber-optic cable into the first and second portions;
couples the first portion into the first spectrometer fiber-optic cable; and
couples the second portion into the second spectrometer fiber-optic cable;
wherein (i) the first spectrometer fiber-optic cable couples the first portion into the first spectrometer and (ii) the second spectrometer fiber-optic cable couples the second portion into the second spectrometer.

43. The system of claim 33, the cassette holder comprising a cuvette holder that positions a cuvette such that the fluid, when confined within the cuvette, is illuminated by the broadband light.

44. The system of claim 43, the cuvette comprising glass or plastic.

45. The system of claim 33, the fluid comprising human saliva.

46. The system of claim 33, the fluid comprising a biological fluid obtained from a human or animal.

47. The system of claim 33, wherein:
the signal processing circuit comprises a memory storing a kernel matrix having a plurality of cells, wherein:
each cell of the plurality of cells is indexed by a first index value of a plurality of first index values and a second index value of a plurality of second index values;
the first index value uniquely identifies one of the first and second pluralities of pixel detectors;
the second index value uniquely identifies one of a plurality of reference wavelengths; and
each cell stores the spectral response of the one of the first and second pluralities of pixel detectors identified by the first index value of said each cell, the spectral response being at the one of the plurality of reference wavelengths identified by the second index value of said each cell; and
the signal processing circuit is further programmed to deconvolve the first and second arrays of raw spectral values by performing least-squares regression based at least on the first and second arrays of raw spectral values and the kernel matrix.

48. The system of claim 47, wherein the signal processing circuit is programmed to perform least-squares regression with Tikhonov regularization.

49. The system of claim 47, wherein:
at least one of the reference wavelengths lies within the first wavelength range; and
at least one of the reference wavelengths lies within the second wavelength range.

50. The system of claim 33, the signal processing circuit comprising a field-programmable gate array.

51. The system of claim 33, the signal processing circuit comprising:
a processor;
a memory communicably coupled to the processor; and
a deconvolution engine implemented as machine-readable instructions that are stored in the memory and, when executed by the processor, control the signal processing circuit to deconvolve the first and second arrays of raw spectral values to obtain the array of deconvolved spectral values.

52. The system of claim 51, wherein:
the memory stores a kernel matrix comprising a plurality of cells, wherein:
each cell of the plurality of cells is indexed by a first index value of a plurality of first index values and a second index value of a plurality of second index values;
the first index value uniquely identifies one of the first and second pluralities of pixel detectors;
the second index value uniquely identifies one of a plurality of reference wavelengths; and
each cell stores the spectral response of the one of the first and second pluralities of pixel detectors identified by the first index value of said each cell, the spectral response being at the one of the plurality of reference wavelengths identified by the second index value of said each cell; and
the machine-readable instructions that, when executed by the processor, control the signal processing circuit to deconvolve include machine-readable instructions that, when executed by the processor, control the signal processing circuit to perform least-squares regression based at least on the first and second arrays of raw spectral values and the kernel matrix.

53. The system of claim 52, wherein the machine-readable instructions that, when executed by the processor, control the signal processing circuit to perform least-squares regression include machine-readable instructions that, when executed by the processor, control the signal processing circuit to perform least-squares regression with Tikhonov regularization.

54. The system of claim 52, wherein:
at least one of the reference wavelengths lies within the first wavelength range; and
at least one of the reference wavelengths lies within the second wavelength range.

55. The system of claim 33, wherein the signal processing circuit is further programmed to:
feed the array of deconvolved spectral values into the trained machine-learning model by transmitting the array of deconvolved spectral values to an external computer system that feeds the array of deconvolved spectral values into the trained machine-learning model to obtain the indication; and
receive the indication from the external computer system.

56. A method comprising:
transmitting broadband light through a fluid confined within a test cassette;
dispersing a first wavelength range of the broadband light, after passing through the test cassette, onto a first plurality of pixel detectors forming a first detector array;
dispersing a second wavelength range of the broadband light, after passing through the test cassette, onto a second plurality of pixel detectors forming a second detector array;
reading a first array of raw spectral values from the first detector array;
reading a second array of raw spectral values from the second detector array;
deconvolving, based on a spectral response of each pixel detector of the first and second pluralities of pixel detectors, the first and second arrays of raw spectral values to generate an array of deconvolved spectral values;
feeding the array of deconvolved spectral values into a trained machine-learning model that processes the array of deconvolved spectral values to obtain an indication of the presence of one or more constituents in the fluid; and
outputting the indication.

57. The method of claim 56,
further comprising merging the first and second arrays of raw spectral values into a composite array of raw spectral values;
wherein said deconvolving comprises deconvolving the composite array of raw spectral values.

58. The method of claim 56, wherein:
said deconvolving comprises:
deconvolving the first array of raw spectral values to obtain a first partial array of deconvolved spectral values; and
deconvolving the second array of raw spectral values to obtain a second partial array of deconvolved spectral values; and
the method further comprises merging the first and second partial arrays of deconvolved spectral values to obtain the array of deconvolved spectral values.

59. The method of claim 56, further comprising collecting the fluid.

60. The method of claim 59, wherein said collecting excludes adding any reagent to the fluid.

61. The method of claim 59, wherein said collecting comprises:
inserting at least part of the fluid into the test cassette; and
placing the test cassette in a cassette holder.

62. The method of claim 59, wherein said collecting comprises collecting the fluid from a human patient.

63. The method of claim 62, wherein said collecting comprises collecting saliva from the human patient.

64. The method of claim 62, further comprising diagnosing, based on the indication, the human patient with a disease.

65. The method of claim 64, further comprising providing the human patient with a therapeutic intervention for treating the disease.

66. The method of claim 65, the therapeutic intervention being a surgical procedure, a non-surgical medical procedure, a prescription for one or more pharmaceutical drugs, or a combination thereof.

67. The method of claim 56, wherein:
said deconvolving comprises performing least-squares regression based at least on the first and second arrays of raw spectral values and a kernel matrix comprising a plurality of cells;
each cell of the plurality of cells is indexed by a first index value of a plurality of first index values and a second index value of a plurality of second index values;
the first index value uniquely identifies one of the first and second pluralities of pixel detectors;
the second index value uniquely identifies one of a plurality of reference wavelengths; and
each cell stores the spectral response of the one of the first and second pluralities of pixel detectors identified by the first index value of said each cell, the spectral response being at the one of the plurality of reference wavelengths identified by the second index value of said each cell.

68. The method of claim 67, wherein said performing least-squares regression comprises performing least-squares regression with Tikhonov regularization.

69. The method of claim 56, wherein said feeding comprises:
transmitting the array of deconvolved spectral values to an external computer system, wherein the external computer system feeds the array of deconvolved spectral values into the trained machine-learning model; and
receiving the indication from the external computer system.

70. A system comprising:
a first light source operable to emit a first broadband light beam;
a second light source operable to emit a second broadband light beam;
a cassette holder that is:
shaped to receive a test cassette that confines a fluid; and
positioned such that the first and second broadband light beams, when emitted by the respective first and second light sources, pass through the test cassette;
a first spectrometer that, in response to receiving the first broadband light beam after the first broadband light beam has passed through the test cassette, disperses the first broadband light beam onto a first plurality of pixel detectors forming a first detector array;
a second spectrometer that, in response to receiving the second broadband light beam after the second broadband light beam has passed through the test cassette, disperses the second broadband light beam onto a second plurality of pixel detectors forming a second detector array;
a digitizer that is electrically connected to the first and second detector arrays, the digitizer being operable to:
read a first array of raw spectral values from the first detector array; and
read a second array of raw spectral values from the second detector array; and
a signal processing circuit programmed to:
deconvolve, based on a spectral response of each of the first and second pluralities of pixel detectors, the first and second arrays of raw spectral values to obtain an array of deconvolved spectral values;
feed the array of deconvolved spectral values into a trained machine-learning model that processes the array of deconvolved spectral values to obtain an indication of the presence of one or more constituents in the fluid; and
output the indication.

71. A method comprising:
constructing a kernel matrix of a spectrometer, wherein:
the kernel matrix comprises a plurality of cells;
each cell of the plurality of cells is indexed by a first index value of a plurality of first index values and a second index value of a plurality of second index values;
the first index value uniquely identifies one of a plurality of pixel detectors of the spectrometer; and
the second index value uniquely identifies one of a plurality of reference wavelengths;
wherein said constructing comprises, for each pixel detector of the plurality of pixel detectors:
constructing a spectral response function based at least on (i) one of a plurality of central wavelengths corresponding to said each pixel detector and (ii) one of a plurality of spectral widths corresponding to said each pixel detector;
evaluating the spectral response function at the plurality of reference wavelengths to obtain a pixel-response vector; and
inserting the pixel-response vector into the kernel matrix.

72. The method of claim 71, further comprising downloading the plurality of reference wavelengths from a memory of the spectrometer.

73. The method of claim 71, further comprising setting all of the plurality of spectral widths to an identical value.

74. The method of claim 71, wherein said constructing the spectral response function comprises constructing a Gaussian function that is centered at said one of the plurality of central wavelengths and has a width based on said one of the plurality of spectral widths.

75. The method of claim 71, further comprising uploading the kernel matrix to a memory of the spectrometer.

76. The method of claim 71, the spectrometer comprising a spectrophotometer.

77. The method of claim 71, wherein the plurality of reference wavelengths are uniformly spaced.

78. A system or method to detect a disease based on spectral properties of a biological fluid without a reagent.
